(19) United States
(12) Patent Application Publication
(10) Pub. No.: US 2023/0277491 A1
(43) Pub. Date: Sep. 7, 2023

(54) PHARMACEUTICAL COMBINATION COMPRISING GLYCOLIC ACID AND L-ALANINE

(71) Applicant: Francisco PAN-MONTOJO, München (DE)

(72) Inventor: Francisco PAN-MONTOJO, Munich (DE)

(21) Appl. No.: 18/018,935

(22) PCT Filed: Jul. 30, 2021
(86) PCT No.: PCT/EP2021/071431

§ 371 Date: Jan. 31, 2023

(30) Foreign Application Priority Data
Jul. 31, 2020 (EP) 20188919.3

(51) Int. Cl. A61K 31/198; A61K 31/19; A61P 15/08; A61P 25/28; A61K 31/575; A61K 31/4415; A61K 31/194; A61K 31/192

(52) U.S. Cl. CPC A61K 31/198; A61K 31/19; A61P 15/08; A61P 25/28; A61K 31/575; A61K 31/4415; A61K 31/194; A61K 31/192

(57) ABSTRACT

A pharmaceutical combination is provided that includes glycolic acid or a pharmaceutically acceptable salt or ester thereof, and L-alanine and/or pyruvate, or a pharmaceutically acceptable salt thereof. A pharmaceutical combination is also provided that includes D-lactate and/or phenylbutyrate and/or tauroursodeoxycholic acid, or pharmaceutically acceptable salts or esters thereof. Methods are provided for use of the pharmaceutical combination in the treatment of neurological medical conditions, for stimulating neuronal plasticity, for regulating intracellular calcium and/or for stimulating mitochondrial function and ATP production, thereby enabling a slowing, reversing and/or inhibiting of the ageing process and/or regulating, preferably stimulating, the immune system.

The invention relates to the field of pharmaceutical combinations and compositions, and combined administration of glycolic acid with additional agents.

The invention therefore relates to a pharmaceutical combination, comprising glycolic acid or a pharmaceutically acceptable salt or ester thereof, and L-alanine and/or pyruvate, or a pharmaceutically acceptable salt thereof. The combination of the invention optionally comprises D-lactate. Further aspects of the invention relate to the combination of the invention for use in the treatment of neurological medical conditions, for stimulating neuronal plasticity, for regulating intracellular calcium and/or for stimulating mitochondrial function and ATP production, thereby enabling a slowing, reversing and/or inhibiting of the ageing process and/or regulating, preferably stimulating, the immune system.

### BACKGROUND OF THE INVENTION

Glycolic acid is known in the art for various uses, such as in the textile industry as a dyeing and tanning agent, in food processing as a flavouring agent and as a preservative, and in the pharmaceutical industry as a skin care agent, in particular as a skin peeling agent. Glycolic acid can also be found in sugar beets, sugarcane and various fruits.

Glycolic acid is well known as a skin treatment agent, for example EP0852946 describes glycolic acid to reduce skin wrinkling, whereas U.S. Pat. No. 5,886,041 describes therapeutic treatments to alleviate cosmetic conditions and symptoms of dermatologic disorders (severe dry skin) with amphoteric compositions containing glycolic acid. EP0906086 describes glycolic acid for topical application as an α-hydroxy acid active ingredient.

Glycolic acid is also known in the context of a polylactic acid-glycolic acid (PLGA) copolymer, which is typically employed as an inert but biologically acceptable carrier material, in which glycolic acid monomers are covalently linked in polymer form. EP2460539 teaches that degradation of the high molecular polymer (PLGA) will not produce free glycolic acid.

Glycolic acid has recently been described as a therapeutic agent for the treatment of neurodegenerative disease (WO 2015/150383), for the enhancement of sperm motility (WO 2016/026843) and for the treatment of ischemic disease (WO 2017/085215). As is described in the prior art, glycolic acid and D-lactate were found to maintain or rescue mitochondrial potential in DJ-1 RNAi depleted HeLa cells with disrupted mitochondrial function, or after in vitro challenge with the toxin paraquat. Following these results, it was found that glycolic acid and D-lactate rescued the survival of dopaminergic neurons after DJ-1 knock-out or under environmental stress, such as paraquat treatment.

Alanine is an α-amino acid that is used in the biosynthesis of proteins. It is non-essential to humans as it can be synthesized metabolically and does not need to be present in the diet. Beta-alanine has been proposed to have some beneficial or protective effect on physical performance and quality of life in Parkinson's Disease (Journal of Exercise Physiology online. 2018 February; 21(1)), working capacity in older adults (Exp Gerontol. 2013 September; 48(9):933-9) or in military performance (Amino Acids. 2015 December; 47(12):2463-74).

Pyruvic acid (CH₃COCOOH) is the simplest of the alpha-keto acids, with a carboxylic acid and a ketone functional group. Pyruvate (the conjugate base, CH₃COCOO—), is a key intermediate in several metabolic pathways throughout the cell. Pyruvic acid can be made from glucose through glycolysis, converted back to carbohydrates (such as glucose) via gluconeogenesis, or to fatty acids through a reaction with acetyl-CoA. It can also be used to construct the amino acid alanine, and as such represents a known precursor for alanine synthesis in the cell.

Despite these recent advances and discoveries regarding the various potential medical applications of glycolic acid, and the potential for employing beta-alanine in aging populations, improvements to the existing therapeutic concepts are required in order to enhance the medical effects of administering glycolic acid.

For example, glycolic acid administration has been linked with potential unwanted side effects when administered at high dosages. For example, the administration of glycolic acid in male Wistar rats lead to the formation of hyperoxaluria and calcium oxalate precipitates both within cortex and medulla of the kidney, indicating a risk of kidney stone formation (World J Nephrol. 2016 Mar. 6; 5(2): 189-194; Clinical Toxicology (2008) 46, 322-324).

The present invention seeks to address these and other disadvantages of the prior art by providing combinations, compositions or other formulations for glycolic acids that potentially alleviate unwanted side effects and enhance therapeutic efficacy.

### SUMMARY OF THE INVENTION

In light of the prior art the technical problem underlying the present invention is to provide alternative or improved means for enhancing or providing novel glycolic acid therapies.

The technical problem underlying the invention may be viewed as the provision of means for reducing unwanted side effects of glycolic acid administration.

The technical problem underlying the invention may be viewed as the provision of means for enhancing the efficacy of glycolic acid in treating neurological medical conditions.

The technical problem underlying the invention may be viewed as the provision of novel means for stimulating neuronal plasticity, stimulating mitochondrial function and ATP production, and/or slowing, reversing and/or inhibiting the ageing process.

These problems are solved by the features of the independent claims. Preferred embodiments of the present invention are provided by the dependent claims.

The invention therefore relates to a pharmaceutical combination, comprising:
a. Glycolic acid or a pharmaceutically acceptable salt or ester thereof, and
b. L-Alanine and/or pyruvate, or a pharmaceutically acceptable salt thereof.

The invention also relates to the combination for use in the treatment of various medical conditions, such as for the treatment and/or prophylaxis of neurological disease, and/or for modulating, preferably enhancing, neuronal plasticity, for regulating intracellular calcium, for stimulating mitochondrial function and ATP production, and/or slowing, reversing and/or inhibiting the ageing process, and corresponding methods of treatment. The invention also relates to the combined administration of glycolic acid (GA) with L-alanine (LA) and/or pyruvate (Pyr) in such treatment.

As demonstrated in more detail below, the combined effect of GA with LA and/or Pyr (GA with LA/Pyr) leads to an unexpected synergistic effect in enhancing the survival of dopaminergic neurons after challenge with paraquat, a known neurotoxin employed as e.g. a Parkinson's model. Paraquat challenge of dopaminergic neurons in vitro leads to severely reduced survival of the cells. The administration LA provides no rescue, and administration of GA provides some rescue. Surprisingly, the combined administration of GA with LA leads to an enhanced rescue, greater than the sum of the effects achieved by either GA and LA alone.

Due to the dopaminergic neurons employed in the experiments described below, the synergies observed appear to translate into clinical settings, providing effective means in treating neurological disease in mammalian, preferably human subjects. Furthermore, this quantitative synergy is evident at multiple concentrations of GA and LA, thereby indicating a general combinatorial enhancement between the two agents.

In some embodiments, based on the surprising finding described herein, the respective doses of GA with LA/Pyr can be reduced compared to usually administered doses. As shown in the examples below, the synergistic effect of the combination of active agents enables lower doses to be administered, for example doses that appear non-efficacious when administered alone show efficacy when administered in the inventive combination. A skilled person could not have derived from common knowledge or the prior art that the inventive combination would allow a more effective and lower dosing of the active agents, thereby potentially maintaining or enhancing efficacy whilst potentially reducing side effects. As is evident from the experimental support provided herein, even low doses of the active agents, for example between 10-50% of the established maximum doses in humans for some active agents, may be employed. Even when administered in such reduced doses, the desired effect of enhanced neuron survival remains greater than the sum of the effects of the individually dosed components, thereby supporting a synergistic effect.

Furthermore, the combined administration of GA with LA/Pyr leads to reduced side effects, in particular with respect to reduced risk of kidney stones and/or reduced kidney or liver function. The use of LA therefore exhibits a double effect, of not only enhancing GA action in enhancing neuron survival, but also reduces and/or prevents and/or reduces the risk of kidney stone formation in a subject receiving GA treatment.

As used herein, L-alanine (LA) and/or pyruvate (Pyr) are considered alternatives that can be combined, if so desired. Pyr is considered a precursor of L-alanine, and therefore may be used in place of or additionally to LA. In some embodiments, the invention therefore relates to the combination of GA and LA or a LA precursor. Pyr is considered, in one embodiment, an LA precursor.

In one embodiment, pyridoxine (Vitamine B6) and/or citrate can be employed (in combination with GA) in addition to LA/Pyr. In one embodiment, pyridoxine (Vitamine B6) and/or citrate can be employed as alternatives to LA/Pyr (in combination with GA).

In some embodiments citrate potassium or salt (inhibits growth of calcium crystals) and/or Allopurinol (reduces formation of oxalate) could also be used to prevent kidney stone formation. Pyridoxine (vitamin B6), a cofactor in the alanine-glycoxylate pathway, may reduce production of oxalate by inducing enzyme activity; in an observational study, high intake of vitamin B6 (>40 mg/day). Therefore, additional factors may be employed to reduce kidney stones (or the risk of kidney stones) that may exist due to GA treatment. These additional factors are preferably LA and/or Pyr, as these compounds not only reduce kidney stones, or risk of developing kidney stones or other kidney malfunction, but show an enhancement of the therapeutic efficacy of GA.

In other embodiments, pyridoxine (Vitamine B6) and/or citrate may be employed in combination or as LA/Pyr alternatives.

Additional beneficial effects can be achieved by the inventive combination and novel uses of GA described herein (either in or independent of the inventive combination).

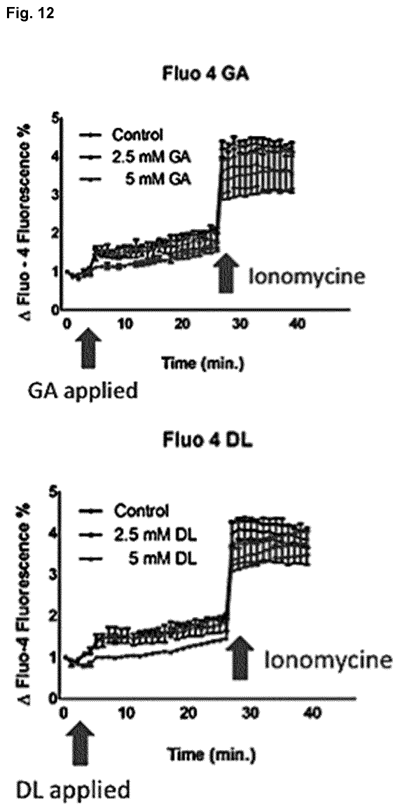

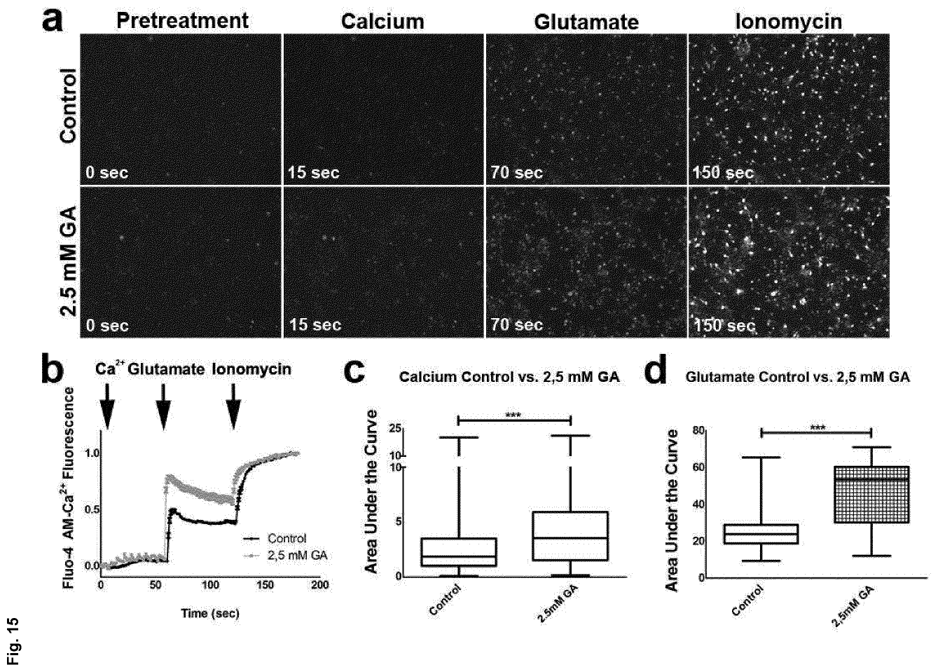

In some embodiments, GA also regulates and/or reduces the levels of intracellular calcium, and this provides a basis for multiple therapeutic effects, as described herein. A direct effect between GA and calcium in the cell is not evident, i.e. the findings of the present invention are not consistent with GA and calcium physically interacting. However, GA can lower intracellular calcium levels, for example in HeLa cells or neurons. The lowering of calcium in the cells allows a greater total calcium influx during stimulation (e.g. upon action potential or initial stages of mitosis). The calcium regulation (lowering intracellular calcium) thereby increases the membrane potential of calcium thereby helping to lower the threshold for an action potential in neurons, and increases calcium influx during action potential (refer FIGS. 12 and 15 below). The effect of GA causes reduced calcium in the cell, but increases storage operated calcium entry, calcium transients and glutamate-dependent calcium entry. This has a positive (therapeutic) effect on neuronal plasticity and long term potentiation. This data is not consistent with the earlier supposition that calcium was physically bound by glycolic acid, the present findings as shown in the examples represent entirely novel and unexpected findings regarding the underlying mechanism and associated therapeutic effects.

This development with respect to combined administration of GA with LA/Pyr therefore exhibits multiple unexpected advantages and enables improved therapeutic regimes.

The combined effects of (a) calcium regulation with (b) mitochondrial energy production, and protection of mitochondrial function, leads to a unique set of effects in the cell that underlies the various therapeutic approaches described herein. As such, the various therapeutic approaches described herein are linked by a unique and unexpected set of functions, thereby establishing a unified set of clinical/medical uses of the inventive combination or GA.

A further potential side effect of GA treatment using high doses is a risk of reactive instant feces deposition (sometimes in fluid form, such as diarrhea). By combining GA with LA/Pyr, the GA dose does not require elevation to a level that may induce such side effects, rather GA can be dosed at a lower level but with good efficacy with respect to e.g. neuron survival.

In one embodiment, the pharmaceutical combination as described herein comprises additionally D-lactate or a pharmaceutically acceptable salt thereof.

Lactic acid is chiral and has two optical isomers; one isomer is L-(+)-lactic acid (LL) and its mirror image, the other isomer, is D-(−)-lactic acid (DL). D- and L-lactic acid are produced naturally by lactic acid bacteria and relatively high levels of D-lactic acid are found in many fermented milk products such as yoghurt and cheese. Of note, no natural product, such as a food product, has sufficient levels to achieve a significant therapeutic effect. Therefore, although e.g. some types of Bulgarian yoghurt has relatively high natural DL levels, these are typically insufficient at their natural levels to achieve a therapeutic effect. In accordance with the present invention, D-lactic acid is known and used as an active ingredient for the treatment of a neurological disease, preferably neurodegenerative disease associated with a decline in mitochondrial activity. L-lactic acid is surprisingly not suitable to treat a neurological disease.

As shown previously, the combined administration of GA and DL can rescue the cell rounding phenotype of DJ-1 mutations and mitochondrial impairment and can stimulate the survival of dopaminergic neurons in vitro and in vivo. In embodiments where the GA and DL are administered at the same time, GA and DL may either be co-formulated before administration or separately administered.

In some embodiments, the pharmaceutical combination of the invention is characterized in that
Glycolic acid is in a pharmaceutical composition in admixture with a pharmaceutically acceptable carrier, and L-alanine and/or pyruvate is in a separate pharmaceutical composition in admixture with a pharmaceutically acceptable carrier, or
Glycolic acid, L-alanine and/or pyruvate, are present in a kit, in spatial proximity but in separate containers and/or compositions, or
Glycolic acid, and L-alanine and/or pyruvate, are combined in a single pharmaceutical composition in admixture with a pharmaceutically acceptable carrier.

As described in detail below the combination of the invention relies on a combined biological effect of the various agents, not on the physical packaging of the agents. Therefore, multiple physical forms of the combination are envisaged, essentially any physical form of the combination is encompassed by the invention with the condition that some interaction or combined biological effect of the agents can be achieved post-administration to a subject.

In some embodiments, the pharmaceutical combination according to the invention is characterized in that a pharmaceutical composition comprising glycolic acid, L-alanine and/or pyruvate is suitable for oral administration to a subject.

Oral administration is a preferred route for administration due to its ease in administration and efficacy observed in human trials. Each of GA, LA and Pyr may be singly prepared in separate oral administration forms, or combined in combination administration forms. Each of GA, LA and Pyr may be prepared in separate and potentially different forms, but all suitable for oral administration, or one or more agents may be suitable for oral administration. For example, GA may be prepared as a solution for oral administration (ingestion), and LA may be prepared as a tablet or oral solid form or ingestion.

In some embodiments, the pharmaceutical combination according to the invention is characterized in that a pharmaceutical composition comprising glycolic acid, L-alanine and/or pyruvate is suitable for injection to a subject.

Injection forms, such as liquids and solutions and the like, may be preferred, depending on the particular condition to be treated. For example, bypassing the GI tract via injection could potentially reduce side effects in some cases. Intrathecal administration could also enhance the amount of agent delivered to the brain.

A preferred mode of administration according to the present invention is transmucosal administration, i.e. through, or across, a mucous membrane. The transmucosal routes of administration of the present invention are preferably intranasal, inhalation, buccal and/or sublingual. Nasal or intranasal administration relates to any form of application to the nasal cavity. The nasal cavity is covered by a thin mucosa which is well vascularized. Therefore, a drug molecule can be transferred quickly across the single epithelial cell layer without first-pass hepatic and intestinal metabolism.

Intranasal administration is therefore used as an alternative to oral administration of for example tablets and capsules, which lead to extensive degradation in the gut and/or liver. Buccal administration relates to any form of application that leads to absorption across the buccal mucosa, preferably pertaining to adsorption at the inside of the cheek, the surface of a tooth, or the gum beside the cheek. Sublingual administration refers to administration under the tongue, whereby the chemical comes in contact with the mucous membrane beneath the tongue and diffuses through it. Inhalation administration is known in the art and typically comprises breathing, or inhaling via an inhaler or other dosage device, an active agent into the lungs, where the active agent enters the blood stream across the lung mucosa.

In some embodiments, transmucosal administration, and especially intranasal administration, have the additional advantage of enabling good transport or delivery of the active agent to the brain, whist avoiding systemic or GI effects. The nasal mucosa is well vascularized and also enables direct/immediate contact with the blood brain barrier, thereby enabling transport of GA to the brain with reduced systemic degradation or side effects.

In some embodiments, the pharmaceutical combination comprises a glycolic acid solution with 5-30 wt % glycolic acid, preferably 15-25 wt % glycolic acid.

These embodiments are preferred as they have been shown to achieve efficacy with respect to the treatment of neurological disease both in vitro and in vivo. The concentrations of glycolic acid differ from those commonly used in topical or cosmetic applications and enable the desired effects when administered, preferably orally or via injection.

In one embodiment, the pharmaceutical combination comprises a GA solution, wherein the glycolic acid solution has a pH of 6-8, preferably about pH 7. The pH range of 6-8 may be considered as essentially neutral. In some embodiments. The pH may be however from 5-9, or any value selected from, or any value in a range of any values selected from, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9 or 9.0.

Adjustment of the pH of the GA solution can be achieved via various means, as known to one skilled in the art, including, without limitation, the use of buffers and/or bases (substances that, when dissolved in water, gives hydroxide ions, OH—, or a species that can accept a proton) to increase pH to an approximately neutral level. In stark contrast to the topical or cosmetic applications of GA, which rely on the low pH of a GA solution to peeling or treat skin, the present invention is based on a therapeutic effect of GA that is independent of the pH of the composition administered. According to the present invention, in preferred embodiments, GA is administered with an essentially neutral or nearly neutral pH, thereby avoiding any unwanted effects due to an acidic pH if GA was administered in solution alone.

Buffers that can be employed for achieving an essentially neutral pH include, without limitation, MES, Bis-Tris, ADA, ACES, PIPES, MOPSO, Bis-Tris Propane, BES, MOPS, TES, HEPES, DIPSO, MOBS, TAPSO, Tris or Trizma®, HEPPSO, POPSO, TEA, EPPS, Tricine, Gly-Gly, Bicine, HEPBS, TAPS, AMPD, TABS, AMPSO, CHES, CAPSO, AMP, CAPS and CABS.

In order to raise the ph level of a glycolic acid solution, to an essentially neutral ph range, various approaches may be employed. For example, alkalizing agents may be used, for example selected from the group consisting of sodium hydroxide, ammonia solution, ammonium carbonate, diethanolamine, potassium hydroxide, sodium bicarbonate, sodium borate, sodium carbonate and trolamine.

In some embodiments, the pharmaceutical combination is characterized in that, (a.) glycolic acid and (b.) L-alanine and/or pyruvate have relative amounts of 1000:1 to 1:100 by weight, preferably 100:1 to 1:10, more preferably about 50:1 to 1:1, more preferably about 5:1 to 1:1, more preferably about 3:1 to 1.5:1.

As described in more detail below, these relative amounts and corresponding dosage regimes enable an effect synergy between the GA and LA/Pyr. Changes in the relative concentrations of the combined agents do not necessarily lead to a loss of synergy when testing the agents at various relative concentrations. As such, the invention encompasses any relative concentration and/or amount of the combined agents disclosed herein.

In some embodiments, the pharmaceutical combination is prepared, configured for administration and/or administered such that:
glycolic acid is administered at a daily dose of greater than 50 mg per kg patient body weight (mg/kg), preferably at a daily dose of 70-150 mg/kg, more preferably at a daily dose of 80-120 mg/kg.

In some embodiments, the pharmaceutical combination is prepared, configured for administration and/or administered such that:
L-alanine is administered at a daily dose of greater than 40 mg per kg patient body weight (mg/kg), preferably at a daily dose of 20-80 mg/kg, more preferably at a daily dose of 30-60 mg/kg.

In some embodiments, GA is administered at 5 to 150 mg/kg to a subject in a daily dose.

In some embodiments, GA is administered at 50, 55, 60, 65, 70, 75, 80, 85, 90, 95, 100, 105, 110, 115, 120, 125, 130, 135, 140, 145, or 150 mg/kg to a subject in a daily dose. Any value similar to these preferred values, or a value falling within a range of any two values from those disclosed, is also encompassed by the present invention.

In some embodiments, GA is administered at 5, 10, 15, 20, 25, 30, 35, 40, 45 or 50 mg/kg to a subject in a daily dose. Any value similar to these preferred values, or a value falling within a range of any two values from those disclosed, is also encompassed by the present invention.

In some embodiments, doses as low as 5 mg/kg GA may be employed. In the case of stroke, as the dose administered intra-arterially is calculated based on the volume of the brain, the total amount given is typically around 1 g, which is, when calculated according to the weight of the whole organism, relatively low.

In some embodiments, LA is administered at 20, 25, 30, 35, 40, 45, 50, 55, 60, 65, 70, 75, or 80 mg/kg to a subject in a daily dose. Any value similar to these preferred values, or a value falling within a range of any two values from those disclosed, is also encompassed by the present invention.

The LA dosages of the invention described herein are surprising, in that they enable the double advantage described herein of reduced kidney side effects and enhanced GA efficacy. It was an unexpected and beneficial finding that even at these low LA levels, no evidence of kidney dysfunction was seen and GA enhancement could be achieved.

In the present application the dose mg/kg relates to amount of active agent per kg body weight of the subject.

By way of example, the following preferred doses are disclosed, that have been assessed in individualized clinical trials.

In human patients, typically when GA is administered in patients in doses lower than 50 mg/kg, the concentration of GA in the blood is too low, and concentrations higher than 150 mg/kg can lead to a reactive instant feces deposition (sometimes in fluid form, which is not desired) and do not increase the concentration of the substances in the blood because the increased intestinal motility does not allow proper absorption. In some cases, reactive instant feces deposition was observed at GA doses above 120 mg/kg, but this upper limit will depend on the particular patient. In some cases, efficacious doses of GA were first observed above 70 mg/kg, but this lower limit will depend on the particular patient.

In human patients, typically 3 to 6 grams per day of L-alanine were employed in treating human subjects, e.g. of 70-80 kg. Therefore between 20 and 80 mg/kg of L-alanine is preferred, more preferred is 30-60 mg/kg in a daily dose of LA. This amount is typically sufficient to prevent any kidney damage or stones, or other renal or liver disfunction.

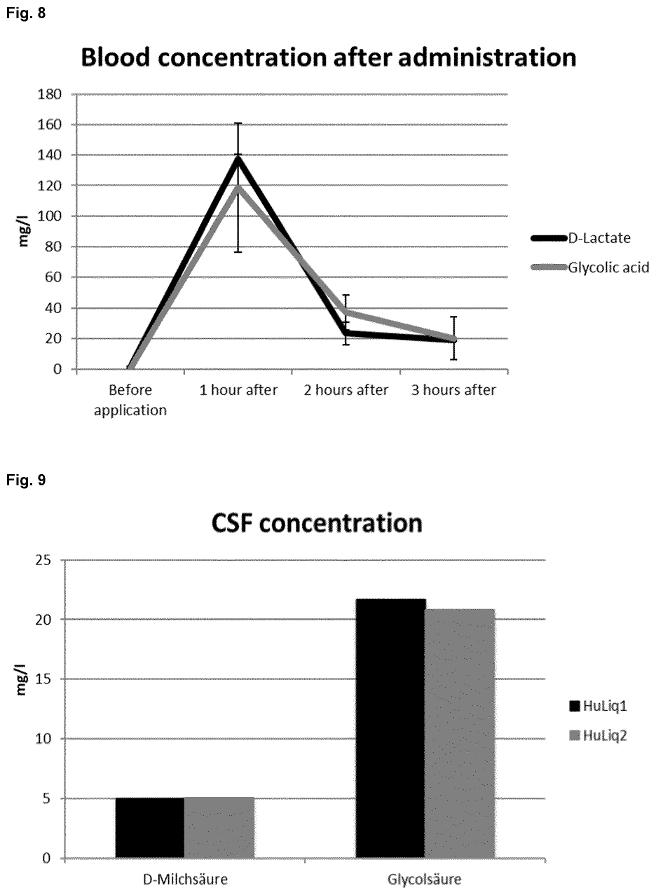

The concentration of GA in the cerebrospinal fluid has been found to be typically about 1:6 lower than in the blood (e.g. 2 mM in the blood and ca. 0.33 mM in the CSF, see FIGS. 8 and 9 below). This concentration is therapeutically relevant to enable clinical efficacy, although this will depend on the indication. 0.33 mM in the CSF appears to be sufficient for some applications (e.g. Parkinson). Other clinical applications may require higher doses (e.g. ALS, stroke), but this remains to be established and the permeability of the blood-brain-barrier in the specific situation should be considered (e.g. in stroke the blood-brain-barrier permeability is increased), and is within the ambit of routine work for a skilled person in testing and achieving a suitable dose.

The different components of this formulation can be mixed together or given separately (e.g. a GA containing solution, and optionally DL, and then L-alanine tablets).

If all compounds are mixed in a solution, the concentration of GA and/or DL in the formulation may, in some embodiments, be between 20% and 50.66%, and the concentration of LA should be between 12.5 and 25.33%.

In one embodiment, an example for a formulation containing 50.66% solution of GA (and optionally DL) and 25.33% of LA:

Add 950 mg/ml of GA, 1.4 grams of sodium DL and 475 mg/ml L-Alanine as powder, then add 7.5M NaOH in such a volume that a concentration of around 50.66% for DL and GA and a concentration of 25.33% of LA with a pH of 6.5 to 7.5 is achieved. By preparing this solution, the osmolality of the solution is minimized, and this reduces any unwanted effects on the intestine. This formulation can then be further diluted in water or e.g. apple juice or supplemented with an additive in order to improve the taste.

For example, a 70 kg patient would, in preferred embodiments, receive as a daily dose between of 5, 6 and 7 grams of GA, between 5, 6 and 7 grams of DL and between 2.1 and 4.2 grams of LA. This means between 5.89 ml and 7.36 ml of the example formulation above.

In other embodiments, formulations based on the combinations of the invention are such that:
1) The end doses to be administered to the patient are between 50 mg/kg, preferably 70 mg/kg but below 150 mg/kg, preferably 120 mg/kg for GA, and between 20 and 80, preferably 30 and 60 mg/kg of LA,
2) Preferably, in some embodiments, the combination is formulated such that the concentration in blood is at least 2 mM for GA (and optionally for DL), preferably 5 mM, and at least 0.01 mM, preferably 0.02 mM for LA.
3) Alternatively, in some embodiments a dose is administered such that the concentration in the cerebrospinal fluid (CSF) is at least 2 mM for GA (and optionally for DL), preferably 5 mM, and at least 0.01 mM, preferably 0.02 mM for LA.
4) Alternatively, in some embodiments a dose is administered such that the concentration in the blood irrigating the affected area is at least 60 mM for GA (and optionally for DL), preferably 120 mM, and at least 0.01 mM, preferably 0.02 mM for LA. This embodiment is an example of, but not limited to, a stroke treatment. And the final amount administered is enough to achieve a concentration of at least 10 mM GA (and optionally DL), preferably 20 mM, and at least 0.01 mM, preferably 0.02 mM for LA, in the target organ.

In one embodiment, the invention relates to a pharmaceutical combination, comprising GA with LA/Pyr, wherein the components are configured for administration or are administered in a dosage or manner sufficient achieve a synergistic effect in protecting and/or rescuing dopaminergic neurons from paraquat challenge in vitro. A skilled person is capable of empirically determining the necessary concentrations, doses and/or relative amounts in order to observe any given synergy. The general disclosure regarding the calculation and assessment of synergistic effects enables a skilled person to determine said concentrations and/or doses without undue effort.

In some embodiments, the pharmaceutical combination is configured for use, or administered such that, a glycolic acid solution is administered intrathecally to a subject.

Intrathecal administration is a route of administration for one or more of the components of the combination via an injection into the spinal canal, or into the subarachnoid space, so that the agent reaches the cerebrospinal fluid (CSF). Intrathecal administration in the present invention represents a preferred embodiment, e.g. for treating neurological conditions, or for increasing neuronal plasticity, as it ensures that the GA, DL, LA and/or Pyr reach the CSF and/or brain.

Considering that CSF levels of GA post-administration are typically about 1:6 lower than in the blood (e.g. 2 mM in the blood and ca. 0.33 mM in the CSF), introducing the GA into the CSF represents a further means of reducing dose and enhancing the efficacy without inducing side effects.

In one embodiment, GA can be administered alone (independent of a combination with DL, LA and/or Pyr) via intrathecal administration.

In a further aspect, the invention therefore relates to glycolic acid or a pharmaceutically acceptable salt or ester thereof, optionally in combination with DL, LA and/or Pyr, for use in the treatment of a neurological medical condition, preferably a neurodegenerative disease, more preferably Amyotrophic Lateral Sclerosis (ALS) or Parkinson's Disease, wherein said treatment comprises the intrathecal administration of glycolic acid or a pharmaceutically acceptable salt or ester thereof.

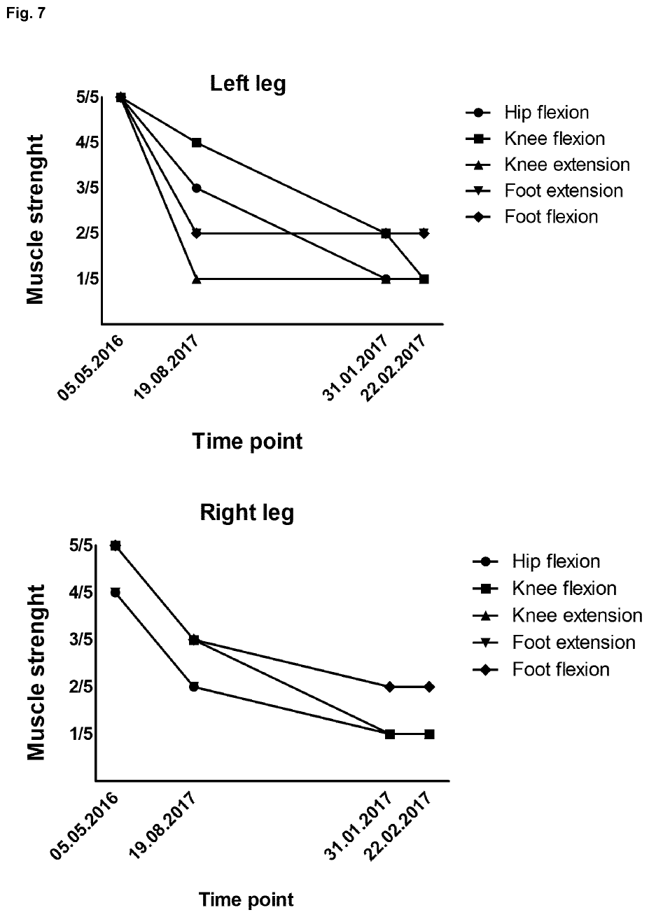

It was a surprising finding of the inventor, that CSF levels of GA post-administration are typically about 1:6 lower than in the blood (e.g. 2 mM in the blood and ca. 0.33 mM in the CSF). This evidence is presented in FIGS. 7 and 8. Therefore, based on this unexpected discovery, introducing GA into the CSF represents improved means of reducing dose and enhancing the efficacy of GA without inducing side effects. To the knowledge of the inventor, no suggestion has been made previously in the art regarding intrathecal administration of GA.

Embodiments of the invention described herein with respect to the inventive combination, also apply to the aspect of the invention regarding administration of GA independent of the combination via intrathecal administration. For example, the concentrations, administration forms, solutions, pH values, doses, and other features of the invention described herein regarding the combination, apply to the intrathecal administration of GA alone (or otherwise independent of the claimed combination), as also described herein.

In some embodiments, the pharmaceutical combination is configured for use, or administered such that, a glycolic acid solution is administered intra-arterially to a subject.

Intra-arterial administration is a route of administration for one or more of the components of the combination via an injection into the artery supplying a certain organ, so that the agent reaches the target organ without going through the lungs and getting diluted. Intra-arterial administration in the present invention represents a preferred embodiment, e.g. for treating ischemia such as stroke, as it ensures that the GA, DL, LA and/or Pyr reach brain-blood-barrier in concentrations high enough to cross it.

In a further aspect, the invention therefore relates to glycolic acid or a pharmaceutically acceptable salt or ester thereof, optionally in combination with DL, LA and/or Pyr, for use in the treatment of a medical condition, preferably an ischemic disease, more preferably stroke, wherein said treatment comprises the intra-arterial administration of glycolic acid or a pharmaceutically acceptable salt or ester thereof in the proximity of the ischemic area at high local concentrations in such a way that the final amount of GA injected enables a final concentration in the area perfused by the artery between 10 and 30 mM, more preferably 15 to 25 mM and most preferably 20 mM.

It was a surprising finding of the inventor, that CSF levels of GA post-administration are typically about 1:6 lower than in the blood (e.g., 2 mM in the blood and ca. 0.33 mM in the CSF). This evidence is presented in FIGS. 7 and 8. Therefore, based on this unexpected discovery, injecting GA intra-arterially in the proximity of the ischemic area in high concentrations with doses calculated on the volume of the target organ represent improved means of reducing dose and enhancing the efficacy of GA without inducing side effects.

For example, an adult male patient with a focal ischemia on one brain hemisphere (volume 0,763 litres) would, in preferred embodiments, receive between of 0,475 and 1.43 grams of GA intra-arterially (between 6.78 and 20.42 mg/kg of body weight in a 70 kg person), diluted in such a concentration and applied with such a flow rate that the final concentration in blood would be between 60 and 180 mM.

In one embodiment, GA can be administered alone (independent of a combination with DL, LA and/or Pyr) via intranasal administration. Intranasal administration is associated with the advantage of good brain transport of an active agent from the nasal cavity to the brain, and potentially enhanced transmission across the blood brain barrier.

In further embodiments of the invention, the pharmaceutical combination described herein is characterized in that each of glycolic acid and L-alanine are administered in single and separate daily doses, within 2 hours of each other, preferably within about 30 minutes of each other. Various modifications of this dosage scheme are envisaged. By way of example, this dosage scheme illustrates that biological relevance and interaction in combination post-administration can be obtained even when the agents of the combination are administered not in admixture but separately but within a short time of each other. Alternative modes of combined administration are described in more detail below.

In a further aspect of the invention, the pharmaceutical combination is intended for use as a medicament, wherein glycolic acid is administered at a daily dose of greater than 120 mg per kg patient body weight (mg/kg), for the treatment of constipation. As described herein, relatively high doses of GA can lead to diarrhea, typically above 120 mg/kg, more preferably above 150 mg/kg GA per day, when administered orally. This observation enables a novel aspect of GA use in a clinical setting.

In a further aspect, the invention relates to the pharmaceutical combination described herein for use in the treatment of a neurological medical condition, preferably a neurodegenerative disease. In a preferred embodiment, the neurological medical condition is a neurodegenerative disease, which is preferably Amyotrophic Lateral Sclerosis (ALS) or Parkinson's Disease. Additional neurological conditions are described at length herein and represent embodiments of the invention.

ALS is preferred and of particular relevance, as individual experimental treatments have demonstrated a therapeutic effect of the treatment and indicate that GA, preferably in the combination described herein, can effectively address ALS pathology and symptoms. Data is presented below.

To date, mutations in more than 30 genes have been linked to the pathogenesis of ALS. Among them, SOD1, FUS and TARDBP are ranked as the three most common genes associated with mutations in ALS. In some embodiments, the ALS patient has one or more mutations in the SOD1, FUS and/or TARDBP genes. The mutations can be screened using standard protocols and are known to a skilled person.

In a further aspect, the invention relates to the pharmaceutical combination described herein for use as a medicament to stimulate neuronal plasticity.

In a further aspect, the invention relates to GA (independent of a combination with DL, LA and/or Pyr) for use as a medicament to stimulate neuronal plasticity.

To the knowledge of the inventors, no mention has been previously made in the prior art regarding an enhancement of neuronal plasticity via GA treatment.

As is disclosed in the examples below, it was surprising to observe that GA reduces intracellular calcium but increases storage operated calcium entry and calcium influx upon certain signals and that it enhances energy production (NAD(P)H) in HeLa cells and neurons. Previous results had only shown a recuperation of the mitochondrial membrane potential during exposure to environmental noxa or in cells and organisms with genetic mutations. Here we show that increases in energy production occur in wild-type cells from basal levels.

Positive trophic effects on neuronal morphology were also observed. In dopaminergic neurons, GA leads to increases in neurite formation with increased length of neurites and axons. Using calcium imaging on cortical neurons, the effect of GA on calcium transients and calcium influx during the action potential was assessed. The examples below show that cortical neurons treated with GA have bigger calcium transients, increased storage operated calcium entry (SOCE) and higher increases in intracellular calcium during the action potential. These increases are due to a higher calcium membrane potential as a result of GA treatment lowering intracellular calcium concentrations. By reducing intracellular calcium, the difference between extracellular and intracellular calcium increases. When the calcium channels open, more calcium flows inside the cell. Altogether, these results suggest that GA could partially revert the effects of aging and enhance neuroplasticity.

The invention therefore relates to methods of enhancing neural plasticity, comprising administering GA, for example in the treatment of psychiatric disorders, such as obsessive-compulsive disorder (OCD), panic disorder, depression, posttraumatic stress disorder (PTSD) and schizophrenia. Preferably, GA enhances neural plasticity in said subjects, thereby enabling other therapeutic approaches, such as psychotherapy, to be more effective.

Based on these observations, in further embodiments the invention relates to the combined use of GA with potentiating the positive effects of psychotherapy. The invention therefore relates to the use of GA for psychotherapy, in particular for the treatment of post-traumatic stress disorder (PTSD), schizophrenia, addiction conditions, depression, and other neurological conditions for which psychotherapy, and enhanced psychotherapy involving enhanced neuroplasticity, is therapeutically relevant.

Several studies have investigated the effect of psychotherapy-like approaches in psychiatric animal models. Extinction of conditioned fear has been successfully used in a post-traumatic stress disorder (PTSD). Extinction of conditioned fear bears resemblance to one form of cognitive therapy, exposure therapy. Additional reports have shown that variations in the expression of Tcf4 lead to a cognition/plasticity phenotype similar to the one observed in schizophrenic patients. Interestingly, these mice also show a higher susceptibility to negative external cues like social defeat and isolation rearing. Putting these mice in an enriched environment (in the case of isolated mice) and increasing handling care (in the case of social defeat) can ameliorate the symptoms caused by both negative cues. Using models such as these, the present invention can demonstrate that GA, optionally in the combination of the invention described herein, can increase neuronal plasticity and thereby potentiate the positive effects of psychotherapy.

Investigations are ongoing with respect to whether glycolic acid and optionally D-lactate, and optionally the combination of the invention, enhance the positive effect of extinction of conditioned fear, enriched environment and increased handling care as psychotherapy-like approaches in the above-mentioned mouse models of PTSD and schizophrenia.

Embodiments of the invention described herein with respect to the inventive combination, also apply to the aspect of the invention regarding administration of GA independent of the combination for stimulating neuroplasticity. For example, the concentrations, administration forms, solutions, pH values, doses, and other features of the invention described herein regarding the combination, apply to the neuronal stimulation via GA alone (or otherwise independent of the claimed combination), as also described herein.

In a further aspect, the invention relates to the pharmaceutical combination described herein for use as a medicament to treat ischemic disease, preferably stroke. As is known for GA treatment, ischemic disease and in particular stroke can be addressed via GA administration. The inventive combination as described herein, can enhance GA efficacy and reduce side effects, and therefore plausibly represents a promising treatment for ischemic disease.

In a further aspect, the invention relates to the pharmaceutical combination described herein for use as a medicament in the treatment and/or prevention of male infertility and/or for enhancing sperm motility. As is known for GA treatment, sperm motility can be enhanced via GA administration. The inventive combination as described herein, can enhance GA efficacy and reduce side effects, and therefore plausibly represents a promising treatment for treating male infertility and/or for enhancing sperm motility.

In a further aspect, the invention relates to the pharmaceutical combination described herein for use as a medicament to stimulate mitochondrial function and ATP production.

In a further aspect, the invention relates to GA (independent of a combination with DL, LA and/or Pyr) for use as a medicament to stimulate mitochondrial function and ATP production.

In a further aspect, the invention relates to the pharmaceutical combination described herein for use in the treatment and/or prevention of an age-related medical condition associated with a decline in mitochondrial function, wherein said treatment and/or prevention comprises slowing, reversing and/or inhibiting the ageing process.

In a further aspect, the invention relates to GA (independent of a combination with DL, LA and/or Pyr) for use in the treatment and/or prevention of an age-related medical condition associated with a decline in mitochondrial function, wherein said treatment and/or prevention comprises slowing, reversing and/or inhibiting the ageing process.

In a further aspect, the invention relates to the pharmaceutical combination described herein for use in stimulating the immune system (e.g. stimulating immune metabolism which has an positive effect on its function) and/or for use in the treatment of a medical condition for which immune stimulation of the immune system is of therapeutic benefit. As used herein, immune system stimulation or immune stimulation relates to an enhancement of the immune system to provide a (wanted) therapeutic benefit.

In a further aspect, the invention relates to GA (independent of a combination with DL, LA and/or Pyr) for use in stimulating the immune system (or immune metabolism which has an positive effect on its function) and/or for use in the treatment of a medical condition for which stimulation of the immune system function is of therapeutic benefit.

In a further embodiment, the invention relates to the pharmaceutical combination described herein for use in regulating a reaction of immune cells which has a positive effect on its function and/or for use in the treatment of a medical condition for which a proper reaction and function of the immune system is of therapeutic benefit. In a further embodiment, the invention relates to GA (independent of a combination with DL, LA and/or Pyr) for use in regulating the reaction of immune cells which has an positive effect on its function and/or for use in the treatment of a medical condition for which a proper reaction and function of the immune system is of therapeutic benefit.

Embodiments of the invention described herein with respect to the inventive combination, also apply to the aspect of the invention regarding administration of GA independent of the combination for stimulating mitochondrial function and ATP production. For example, the concentrations, administration forms, solutions, pH values, doses, and other features of the invention described herein regarding the combination, apply to the stimulating of mitochondrial function and ATP production via GA alone (or otherwise independent of the claimed combination), as also described herein. These embodiments also apply to the aspects regarding slowing, reversing and/or inhibiting the ageing process and/or stimulating the immune system.

As described in more detail below, modifying the mitochondrial function and enhancing ATP production via GA treatment enables various biological and clinical applications of GA as an active agent. By stimulating ATP production, the immunometabolism is enhanced, thereby enabling the employment of, or incorporation of, GA into new or existing immune treatments. Stimulating mitochondrial function also leads to anti-ageing applications.

For example, it has been shown that that T cells with dysfunctional mitochondria act as accelerators of senescence. In mice, these cells instigate multiple aging-related features, including metabolic, cognitive, physical, and cardiovascular alterations, which together result in premature death. T cell metabolic failure induces the accumulation of circulating cytokines, which resembles the chronic inflammation that is characteristic of aging (“inflammaging”). This cytokine storm itself acts as a systemic inducer of senescence.

Others have shown that among diverse factors that contribute to human aging, the mitochondrial dysfunction has emerged as one of the key hallmarks of aging process and is linked to the development of numerous age-related pathologies including metabolic syndrome, neurodegenerative disorders, cardiovascular diseases and cancer. Mitochondria are central in the regulation of energy and metabolic homeostasis, and harbor a complex quality control system that limits mitochondrial damage to ensure mitochondrial integrity and function (reviewed in The Mitochondrial Basis of Aging and Age-Related Disorders Sarika Srivastava, Genes, 2017)

Additionally, the regulation of calcium homeostasis through GA could be beneficial to obtain a proper reaction of the immune system. Several studies have shown that in cells of the immune system, calcium signals are essential for diverse cellular functions including differentiation, effector function and gene transcription. After engagement of immunoreceptors such as T-cell and B-cell antigen receptors and the Fc receptors on mast cells and NK cells, “store-operated” Ca2+ entry constitutes the major pathway of intracellular Ca2+ increase (reviewed in “Calcium signaling in lymphocytes” Masatsugu Oh-hora and Anjana Rao, Current Opinion in Immunology 2008, https://www.ncbi.nlm.nih.gov/pmc/articles/PMC2574011/)

In a further aspect, the invention relates to the pharmaceutical combination described herein for use in the treatment and/or prevention of alterations in embryonic development associated with a decline in storage associated calcium entry during mitosis and a decline in mitochondrial function, wherein said treatment and/or prevention comprises enhancing or supporting embryonic development during pregnancy or in vitro.

In a further aspect, the invention relates to GA (independent of a combination with DL, LA and/or Pyr) for use in the treatment and/or prevention of alterations in embryonic development associated with a decline in storage associated calcium entry during mitosis and a decline in mitochondrial function, wherein said treatment and/or prevention comprises enhancing or supporting embryonic development during pregnancy or in vitro.

In a further aspect, the invention relates to the pharmaceutical combination as described herein for use as a medicament to stimulate oocyte and fertility fitness.

In a further aspect, the invention relates to the pharmaceutical combination as described herein for use in the treatment and/or prevention of disease- or age-related reduction in fertility in woman.

As described in more detail below GA increases calcium entry during mitosis. Several studies have investigated the role of calcium influx during mitosis and it has been reported that calcium influx is important during mitosis. Surprisingly, our studies showed that knocking down PARK-7 results in a decreased calcium entry during mitosis and in a reduced cell proliferation in HeLa cells. Knocking down PARK-7 or GLO-4 also results in a reduced breed size in mice and a reduced brood size in C. elegans. We also show that this effect is a result of decreased fertility rates and increased abortion rates. Therefore we tested the effect of GA on rescuing cell proliferation and brood size in C. elegans. Our results show that GA is able to rescue these phenotypes.

In a further embodiment, the pharmaceutical combination as described herein comprises additionally 4-phenylbutyric acid (PB) or a pharmaceutically acceptable salt or ester thereof.

In a further embodiment, the pharmaceutical combination as described herein comprises additionally D-lactate and 4-phenylbutyric acid (PB) or a pharmaceutically acceptable salt or ester thereof.

4-Phenylbutyric acid (PB) is an aromatic acid. Sodium phenylbutyrate is used in the treatment of urea cycle disorders, protein misfolding diseases or neurodegenerative diseases. According to several studies, the protective effect in models of neurodegenerative diseases is mediated by an increase in the expression of DJ-1, a Parkinson disease related gene, and protect cells against endogenous or environmental toxins.

As demonstrated in more detail below, PB exerted certain protection against 12.5 μM paraquat. Surprisingly, adding GA leads to an unexpected synergistic effect in enhancing the survival of dopaminergic neurons after challenge with paraquat, a known neurotoxin employed as e.g. a Parkinson's model. Paraquat challenge of dopaminergic neurons in vitro leads to severely reduced survival of the cells. The administration of up to 0.15 mM of PB provides certain protection, and administration of 3 mM of GA provides some rescue. Surprisingly, the combined administration of GA with PB leads to an enhanced rescue, greater than the sum of the effects achieved by either GA or PB alone.

It was surprising that glycolic acid enhanced the effect of PB because: i) GA has no known effect on DJ-1 expression and ii) if PB enhances DJ-1 (which reduces glyoxal and methyglyoxal and increases GA and DL) it would be surprising that further adding GA above physiological levels would have an additional synergistic effect.

Due to the dopaminergic neurons employed in the experiments described below, the synergies observed provide a sound basis to translate into clinical settings, providing effective means in treating neurological disease in mammalian, preferably human subjects. Furthermore, this quantitative synergy is evident at multiple concentrations of GA and PB, thereby indicating a general combinatorial enhancement between the two agents.

In some embodiments, based on the surprising finding described herein, the respective doses of GA with PB can be reduced compared to usually administered doses. As shown in the examples below, the synergistic effect of the combination of active agents enables lower doses to be administered, for example doses that appear non-efficacious when administered alone show efficacy when administered in the inventive combination. A skilled person could not have derived from common knowledge or the prior art that the inventive combination would allow a more effective and lower dosing of the active agents, thereby potentially maintaining or enhancing efficacy whilst potentially reducing side effects. As is evident from the experimental support provided herein, even low doses of the active agents, for example between 10-50% of the established maximum doses in humans for some active agents, may be employed. Even when administered in such reduced doses, the desired effect of enhanced neuron survival remains greater than the sum of the effects of the individually dosed components, thereby supporting a synergistic effect.

In a further embodiment, the pharmaceutical combination as described herein comprises additionally tauroursodeoxycholic acid (TUDCA) or a pharmaceutically acceptable salt or ester thereof.

In a further embodiment, the pharmaceutical combination as described herein comprises additionally D-lactate and tauroursodeoxycholic acid (TUDCA) or a pharmaceutically acceptable salt or ester thereof.

Tauroursodeoxycholic acid is an ambiphilic bile acid. Ongoing research has shown that TUDCA has diminishing apoptotic effects, with potential application in heart disease, Huntington's disease, Parkinson's disease, amyotrophic lateral sclerosis and stroke.

In a further embodiment, the pharmaceutical combination as described herein comprises additionally 4-phenylbutyric acid (PB) or a pharmaceutically acceptable salt or ester thereof and tauroursodeoxycholic acid (TUDCA) or a pharmaceutically acceptable salt or ester thereof.

In a further embodiment, the pharmaceutical combination as described herein comprises additionally D-lactate and 4-phenylbutyric acid (PB) or a pharmaceutically acceptable salt or ester thereof and tauroursodeoxycholic acid (TUDCA) or a pharmaceutically acceptable salt or ester thereof.

The combination of PB and TUDCA has shown to slow down the progression of the disease in ALS patients by approximately 25%. According to several studies, this effect is mediated by a reduction of ER stress and the improvement of the mitochondrial activity. As demonstrated in more detail below the combination of PB and TUDCA did not exert any protection against 12.5 μM paraquat. Surprisingly, substituting PB in this formulation by GA leads to an unexpected synergistic effect with TUDCA in enhancing the survival of dopaminergic neurons after challenge with paraquat, a known neurotoxin employed as e.g. a Parkinson's model. Paraquat challenge of dopaminergic neurons in vitro leads to severely reduced survival of the cells. The administration of the combination of PB and TUDCA provides no rescue, the administration of 1 mM or 3 mM of GA provides no rescue and the administration of 5 mM GA provides certain rescue. Surprisingly, the combined administration of GA with TUDCA leads to an enhanced rescue, greater than the effect of PB and TUDCA in combination.

Due to the dopaminergic neurons employed in the experiments described below, the synergies observed appear to translate into clinical settings, providing effective means in treating neurological disease in mammalian, preferably human subjects. Furthermore, this quantitative synergy is evident at multiple concentrations of GA and TUDCA, thereby indicating a general combinatorial enhancement between the two agents.

In some embodiments, based on the surprising finding described herein, the respective doses of GA with TUDCA can be reduced compared to usually administered doses. As shown in the examples below, the synergistic effect of the combination of active agents enables lower doses to be administered, for example doses that appear non-efficacious when administered alone show efficacy when administered in the inventive combination. A skilled person could not have derived from common knowledge or the prior art that the inventive combination would allow a more effective and lower dosing of the active agents, thereby potentially maintaining or enhancing efficacy whilst potentially reducing side effects. As is evident from the experimental support provided herein, even low doses of the active agents, for example between 10-50% of the established maximum doses in humans for some active agents, may be employed. Even when administered in such reduced doses, the desired effect of enhanced neuron survival remains greater than the sum of the effects of the individually dosed components, thereby supporting a synergistic effect.

In a further aspect of the invention, the pharmaceutical combination comprises GA or a pharmaceutically acceptable salt or ester thereof and 4-phenylbutyric acid or a pharmaceutically acceptable salt or ester thereof.

In a further aspect of the invention, the pharmaceutical combination comprises GA or a pharmaceutically acceptable salt or ester thereof and tauroursodeoxycholic acid (TUDCA) or a pharmaceutically acceptable salt or ester thereof.

In a further aspect the pharmaceutical combination comprises GA or a pharmaceutically acceptable salt or ester thereof and 4-phenylbutyric acid or a pharmaceutically acceptable salt or ester thereof and tauroursodeoxycholic acid or a pharmaceutically acceptable salt or ester thereof.

These aspects of the invention are independent from the use of L-alanine or pyruvate, although L-alanine or pyruvate can be combined in these aspects if so desired. The remaining features of the invention with respect to GA formulation and/or administration also apply to aspects of the invention related to GA and PB, GA and TUDCA, and/or GA, PB and TUDCA.

The features of the invention relating to the pharmaceutical combination also relate to the composition, and vice versa, and to the methods of treatment or indicated medical uses as described herein. Any reference to GA, LA, Pyr, DL, PB or TUDCA is considered to include reference to a pharmaceutically acceptable salt or ester thereof, even if not explicitly mentioned.

### DETAILED DESCRIPTION OF THE INVENTION

Pharmaceutical Combination:

According to the present invention, a “pharmaceutical combination” is the combined presence of glycolic acid with L-alanine and/or pyruvate, i.e. in proximity to one another. In one embodiment, the combination is suitable for combined administration.

In one embodiment, the pharmaceutical combination as described herein is characterized in that GA is in a pharmaceutical composition in admixture with a pharmaceutically acceptable carrier, and LA/Pyr is in a separate pharmaceutical composition in admixture with a pharmaceutically acceptable carrier. The pharmaceutical combination of the present invention can therefore in some embodiments relate to the presence of two separate compositions or dosage forms in proximity to each other. The agents in combination are not required to be present in a single composition or packaging.

In one embodiment, the pharmaceutical combination as described herein is characterized in that GA and LA/Pyr are present in a kit, in spatial proximity but in separate containers and/or compositions. The production of a kit lies within the abilities of a skilled person. In one embodiment, separate compositions comprising two separate agents may be packaged and marketed together as a combination. In other embodiments, the offering of the two agents in combination, such as in a single catalogue, but in separate packaging is understood as a combination.

In one embodiment, the pharmaceutical combination as described herein is characterized in that GA and LA/Pyr are combined in a single pharmaceutical composition in admixture with a pharmaceutically acceptable carrier. Combination preparations or compositions are known to a skilled person, who is capable of assessing compatible carrier materials and formulation forms suitable for both agents in the combination.

Glycolic Acid:

Glycolic acid (GA) has the IUPAC name 2-hydroxyethanoic acid and the molecular formula C2H4O3. Glycolic acid is used in the prior art, for example, in the textile industry as a dyeing and tanning agent, in food processing as a flavouring agent and as a preservative, and in the pharmaceutical industry as a skin care agent, in particular as a skin peeling agent. Glycolic acid can also be found in sugar beets, sugarcane and various fruits. Traces of glycolic acid are present, for example, in unripe or green grapes. Glycolic acid is also found in pineapple and cantaloupe.

A pharmaceutically acceptable salt of glycolic acid includes but is not limited to potassium glycolate, sodium glycolate, calcium glycolate, magnesium glycolate, barium glycolate, aluminium glycolate, oxalate, nitrate, sulphate, phosphate, fumarate, succinate, maleate, besylate, tosylate, tartrate, and palmitate. The production of salts of glycolic acid and the necessary acids used during productions of said salts are within the capabilities of a skilled person.

A pharmaceutically acceptable ester of glycolic acid includes but is not limited to methyl glycolate, ethyl glycolate, butyl glycolate, lauryl glycolate, piperidyl(2)-glycolic acid ethyl, (3-thienyl)-glycolic acid, myristyl glycolate, quinolyl glycolate and cetyl glycolate. Ester compounds of GA may be determined and synthesized by a skilled person as is required without undue effort. In some embodiments the ester is intended to enable cleavage of the ester in vivo, thereby releasing GA as the active component.

Glycolic acid (GA) is naturally present in a variety of fruits, vegetables, meats and beverages, however in amount being lower than 50 mg/kg. 50 mg/kg correspond to 0.005% (w/w). Hence, the formulation of the invention preferably comprises a higher amount/concentration of glycolic acid or a corresponding pharmaceutically acceptable salt or ester thereof than the amount of glycolic acid found in natural food.

The skilled person can determine a suitable dose of such formulations as well as a suitable dosage in case glycolic acid or a pharmaceutically acceptable salt or ester thereof are directly administered to a subject. The administered amounts of glycolic acid or a pharmaceutically acceptable salt or ester thereof on the one hand have to be sufficient for the treatment or prevention of the medical condition, and on the other hand should not be so high as to generate an acidosis in the subject to be treated. Acidosis is an increased acidity in the blood and other body tissue. Acidosis is said to occur when the blood, serum or body tissue pH falls below 7.35. Means and methods to determine the pH in blood, serum and body tissue are well-known. Suitable doses will be discussed herein below.

The toxic effect of too much glycolic acid is known, for example, from the 1985 diethylene glycol wine scandal. The scandal involved a limited number of Austrian wineries that had illegally adulterated their wines using the toxic substance diethylene glycol (a primary ingredient in some brands of antifreeze) to make the wines appear sweeter and more full-bodied. The major cause of toxicity is not the ethylene glycol itself but its major metabolite glycolic acid. The minimum toxic dose of diethylene glycol is estimated at 0.14 mg glycolic acid per kg of body weight and the lethal dose is estimated between 1.0 and 1.63 g/kg.

L-Alanine:

Alanine (symbol Ala or A) is an α-amino acid that is used in the biosynthesis of proteins. It contains an amine group and a carboxylic acid group, both attached to the central carbon atom which also carries a methyl group side chain. Consequently, its IUPAC systematic name is 2-aminopropanoic acid, and it is classified as a nonpolar, aliphatic α-amino acid. Under biological conditions, it exists in its zwitterionic form with its amine group protonated (as —NH3+) and its carboxyl group deprotonated (as —CO2−). It is non-essential to humans as it can be synthesised metabolically and does not need to be present in the diet.

The L-isomer of alanine (left-handed) is the one that is incorporated into proteins. L-Alanine is second only to leucine in rate of occurrence, accounting for 7.8% of the primary structure in a sample of 1,150 proteins. The right-handed form, D-alanine, occurs in polypeptides in some bacterial cell walls and in some peptide antibiotics.

Pyruvate:

Pyruvate has the molecular formula CH3COCOO— and the IUPAC name 2-oxopropanoic acid salt. Pyruvate supplies energy to living cells through the citric acid cycle (also known as the Krebs cycle) when oxygen is present (aerobic respiration), and alternatively ferments to 30 produce lactic acid when oxygen is lacking (fermentation). Tanaka et al. (2007), Mitochondrion, 7(6):399-401, for example, describes the therapeutic potential of pyruvate therapy for mitochondrial diseases. Pyruvate can also be used to construct the amino acid alanine, and as such represents a well-known precursor for alanine synthesis in the cell. Without being bound by theory, partly for this reason, L-alanine and pyruvate are often disclosed as alternatives (or potentially combined) in in the combination of the invention.

Combining pyruvate and/or L-alanine, with glycolic acid and a pharmaceutically acceptable salt or ester thereof, (and optionally with D-lactic acid or a pharmaceutically acceptable salt or ester thereof) can be expected to have an additive beneficial or preferably synergistic effect in the biological effects described herein.

D-Lactate/Lactic Acid:

In one embodiment of the invention the combination described herein is characterised in that D-Lactate or a pharmaceutically acceptable salt thereof is present. A pharmaceutically acceptable ester of lactic acid includes but is not limited to methyl lactate or ethyl lactate.

Lactic acid has the IUPAC name 2-hydroxypropanoic acid and the molecular formula C3H603. Lactic acid is found primarily in sour milk products, such as yogurt, buttermilk, kefir, some cottage cheeses and kombucha but also, for example, in pickled vegetables, and cured meats and fish. As a food additive it is, for example, approved for use in the EU, US, Australia, and New Zealand. Lactic acid is furthermore listed by its INS number 270 or as E number E270. Lactic acid is used in the art as a food preservative, curing agent, and flavouring agent. It is an ingredient in processed foods and is used as a decontaminant during meat processing.

Lactic acid is chiral and has two optical isomers. One isomer is L-(+)-lactic acid (LL) or (Sy lactic acid, and its mirror image, the other isomer, is D-(−)-lactic acid (DL) or (R)-lactic acid. D- and L-lactic acid are produced naturally by lactic acid bacteria. High level of D-lactic acid is found in many fermented milk products such as yoghurt and cheese. In accordance with the present invention D-lactic acid is used as active ingredient in the combination of the invention.

4-Phenylbutyric Acid:

In one embodiment of the invention the combination described herein is characterised in that 4-Phenylbutyric acid or a pharmaceutically acceptable salt or ester thereof is present.

A pharmaceutically acceptable salt of 4-Phenylbutyric acid includes but is not limited to potassium phenylbutyrate (PB), sodium phenylbutyrate, calcium phenylbutyrate, magnesium phenylbutyrate, barium phenylbutyrate, aluminium phenylbutyrate, oxalate, nitrate, sulphate, phosphate, fumarate, succinate, maleate, besylate, tosylate, tartrate, and palmitate. The production of salts of 4-phenylbutyric acid and the necessary acids used during productions of said salts are within the capabilities of a skilled person.

A pharmaceutically acceptable ester of 4-phenylbutyric acid includes but is not limited to methyl phenylbutyrate, ethyl phenylbutyrate, butyl phenylbutyrate, lauryl phenylbutyrate, piperidyl(2)-4-phenylbutyric acid ethyl, (3-thienyl)-4-phenylbutyric acid, myristyl phenylbutyrate, quinolyl phenylbutyrate and cetyl phenylbutyrate. Ester compounds of PB may be determined and synthesized by a skilled person as is required without undue effort. In some embodiments the ester is intended to enable cleavage of the ester in vivo, thereby releasing PB as the active component.

4-Phenylbutyric acid is an aromatic acid made up of an aromatic ring and butyric acid. 4-Phenylbutyric acid has the IUPAC name 3-phenylbutanoic acid and the molecular formula C10H12O2. It's salt, PB is a chemical derivative of butyric acid naturally produced by colonic bacteria fermentation. Phenylbutyrate displays potentially favorable effects on many pathologies including cancer, genetic metabolic syndromes, neuropathies, diabetes, hemoglobinopathies, and urea cycle disorders. 4-Phenylbutyric acid is a human metabolite and is given as a prodrug. In the human body it is first converted to phenylbutyryl-CoA and then metabolized by mitochondrial beta-oxidation, mainly in the liver and kidneys, to the active form, phenylacetate. Phenylacetate conjugates with glutamine to phenylacetylglutamine, which is eliminated with the urine. It contains the same amount of nitrogen as urea, which makes it an alternative to urea for excreting nitrogen.

A 5 g tablet or powder of sodium phenylbutyrate taken by mouth can be detected in the blood within 15 minutes and reaches peak concentration in the bloodstream within an hour. It is metabolized into phenylacetate within half an hour. In the cells, it functions as a histone deacetylase inhibitor and chemical chaperone, leading respectively to research into its use as an anti-cancer agent and in protein misfolding diseases such as cystic fibrosis or neurodegenerative diseases.

Tauroursodeoxycholic Acid (TUDCA):

In one embodiment of the invention the combination described herein is characterised in that tauroursodeoxycholic acid or a pharmaceutically acceptable salt or ester thereof is present.

Tauroursodeoxycholic acid is a bile acid taurine conjugate derived from ursoodeoxycholic acid. Tauroursodeoxycholic acid has the IUPAC name 2-[[(4R)-4[(3R,5S,7S,8R,9S,10S,13R,14S,17R)-3,7-dihydroxy-10,13-dimethyl-2,3,4,5,6,7,8,9,11,12,14,15,16,17-tetradecahydro-1H-cyclopenta[a]phenanthren-17-yl]pentanoyl]amino]ethanesulfonic acid and the molecular formula C26H45NO6S. It is also known as taurursodiol. It has a role as a human metabolite, an anti-inflammatory agent, a neuroprotective agent, an apoptosis inhibitor, a cardioprotective agent and a bone density conservation agent. It derives from an ursodeoxycholic acid. It is a conjugate acid of a tauroursodeoxycholate. Tauroursodeoxycholic acid is the more hydrophilic form of ursodeoxycholic acid, which is the more abundant naturally produced bile acid in humans. Tauroursodeoxycholic acid, on the other hand, is produced abundantly in bears and has been used for centuries as a natural remedy in some Asian countries. It is approved in Italy and Turkey for the treatment of cholesterol gallstones and is an investigational drug in China, Unites States, and Italy. Tauroursodeoxycholic acid is being investigated for use in several conditions such as Primary Biliary Cirrhosis (PBC), insulin resistance, amyloidosis, Cystic Fibrosis, Cholestasis, and Amyotrophic Lateral Sclerosis.

A pharmaceutically acceptable salt of tauroursodeoxycholic acid includes but is not limited to tauroursodeoxycholic acid sodium salt, tauroursodeoxycholic acid potassium salt, tauroursodeoxycholic acid calcium salt, tauroursodeoxycholic acid magnesium salt, tauroursodeoxycholic acid barium salt, tauroursodeoxycholic acid aluminium salt, oxalate, nitrate, sulphate, phosphate, fumarate, succinate, maleate, besylate, tosylate, tartrate, and palmitate. The production of salts of tauroursodeoxycholic acid and the necessary acids used during productions of said salts are within the capabilities of a skilled person.

A pharmaceutically acceptable ester of tauroursodeoxycholic acid includes but is not limited to N-ethyl-tauroursodeoxycholic acid, N-methyl tauroursodeoxycholic acid, N-butyl tauroursodeoxycholic acid, lauryl tauroursodeoxycholic acid, piperidyl(2)-tauroursodeoxycholic acid ethyl, (3-thienyl)-tauroursodeoxycholic acid, myristyl tauroursodeoxycholic acid, quinolyl tauroursodeoxycholic acid and cetyl tauroursodeoxycholic acid. Ester compounds of tauroursodeoxycholic acid may be determined and synthesized by a skilled person as is required without undue effort. In some embodiments the ester is intended to enable cleavage of the ester in vivo, thereby releasing tauroursodeoxycholic acid as the active component.

TUDCA prevents apoptosis with its role in the BAX pathway. BAX, a molecule that is translocated to the mitochondria to release cytochrome C, initiates the cellular pathway of apoptosis. TUDCA prevents BAX from being transported to the mitochondria. This protects the mitochondria from perturbation and the activation of caspases. TUDCA also acts as a chemical chaperone. Recently, TUDCA has been found to have protective effects in the eye, especially concerning retinal degenerative disorders.

Additional Optional Components of the Combination and/or Composition:

Citric acid is a weak organic acid that has the chemical formula C6H8O7. It occurs naturally in citrus fruits. In biochemistry, it is an intermediate in the citric acid cycle, which occurs in the metabolism of all aerobic organisms. A citrate is a derivative of citric acid; that is, the salts, esters, and the polyatomic anion found in solution. When part of a salt, the formula of the citrate anion is written as C6H5O7. Citrate prevents kidney stone formation, and is assumed to act via two mechanisms. It binds with urinary calcium, thereby reducing the supersaturation of urine. In addition, it binds calcium oxalate crystals and prevents crystal growth.

Pyridoxine, also known as vitamin B6, is a form of vitamin B6 found commonly in food and used as dietary supplement. It is required by the body to make amino acids, carbohydrates, and lipids. Sources in the diet include fruit, vegetables, and grain. It is also required for muscle phosphorylase activity associated with glycogen metabolism. Vitamin B6 (pyridoxine) intake can lower the urinary excretion of oxalate, which in turn is one of the major determinants of calcium oxalate kidney stones.

Vitamin E (tocopherol) and vitamin C (ascorbic acid) are antioxidants and are therefore used in the art in the therapy of mitochondrial diseases. In more detail, accumulation of free radicals may be especially harmful to mitochondrial disease patients. The use of antioxidants, like Vitamin C and Vitamin E can help to reduce free radical accumulation, which at least in some patients may mean improvements in energy and function (see Parikh et al. (2009), Current Treatment Options in Neurology, 11:414-430).

B vitamin 2 (B2, Ribofavin) is a water-soluble vitamin that serves as a flavoprotein precursor. It is a key building block in complex I and II and a cofactor in several other key enzymatic reactions involving fatty acid oxidation and the Krebs cycle. Several non-randomized studies have shown vitamin B2 to be efficacious in treating mitochondrial diseases, in particular complex I and/or complex II disease (see Parikh et al. (2009), Current Treatment Options in Neurology, 11:414-430).

Arginine is a semi-essential amino acid involved in growth, urea detoxification, and creatine synthesis. L-arginine produces nitric oxide, which has neurotransmitter and vasodilatory properties (see Parikh et al. (2009), Current Treatment Options in Neurology, 11:414-430).

L-carnitine is a cellular compound that plays a critical role in the process of mitochondrial Carnitine transfers long-chain fatty acids across the mitochondria inner membrane as acylcarnitine esters. These esters are oxidized to acetyl CoA, which enters the Krebs cycle and results in subsequent generation of ATP via oxidative phosphorylation (see Parikh et al. (2009), Current Treatment Options in Neurology, 11:414-430).

Creatine, a compound present in cells, combines with phosphate in the mitochondria to form phosphocreatine. It serves as a source of high-energy phosphate, released during anaerobic metabolism. It also acts as an intracellular buffer for ATP and as an energy shuttle for the movement of high-energy phosphates from mitochondrial sites of production to cytoplasmic sites of utilization. The highest concentrations of creatine are found in tissues with high energy demands, such as skeletal muscle and brain. Creatine is continuously replaced through a combination of diet and endogenous synthesis (see Parikh et al. (2009), Current Treatment Options in Neurology, 11:414-430).

L-arginine, L-carnitine and L-creatine are currently used for the treatment of mitochondrial diseases; see for review Parikh et al. (2009), Current Treatment Options in Neurology, 11:414-430. Hence, combining L-arginine, L-carnitine and/or L-creatine with glycolic acid and a pharmaceutically acceptable salt or ester thereof can be expected to have an additive beneficial or preferably synergistic effect in the treatment of a neurodegenerative disease which is associated with a decline in mitochondrial activity.

In one embodiment of the invention, in addition, one or more of L-arginine, L-carnitine and L-creatine is/are used for the treatment of said disease which is associated with a decline in mitochondrial activity. A formulation in accordance with this preferred embodiment may comprise glycolic acid and a pharmaceutically acceptable salt or ester thereof and in addition one or more of L-arginine, L-carnitine and/or L-creatine, and optionally one or more of pyruvate, one or more of D-lactate, one or more antioxidants and/or one or more vitamins, such as vitamin E, vitamin C and/or B vitamin 2.

Buffers/pH Regulation:

For preparations that are intended to be applied to the sensitive membranes of the eye or nasal passages or that may be injected into muscles, blood vessels, organs, tissue, or lesions, it is desirable to adjust the pH of the preparation to a level that is close to the physiologic pH of the tissue. This is typically done to minimize tissue damage and pain, or discomfort experienced by the patient. First, the route of administration for the dosage form is often considered in selecting appropriate buffers or pH values. Ingredients to buffer or adjust pH must be nontoxic for the intended route of administration. This is an important factor to consider. For example, boric acid and sodium borate are common ingredients for ophthalmic solutions; these would not be satisfactory for systemic drug preparations because borate is toxic systemically. Agents for any route of administration should be nonirritating at the needed concentration. For oral liquid preparations, buffer compounds should preferably not have a disagreeable odor or taste. Agents used for parenteral preparations must be in sterile form or must be rendered sterile.

If a formula calls for the adjustment of pH to a given level, usually a dilute solution (0.1 to 0.2 N) of HCl or NaOH may be used. Sodium Bicarbonate may be used to raise the pH of preparations. It is sterile and nontoxic. For oral or topical liquids, a preformulated vehicle may be used. Many of the available flavored syrups and liquid vehicles contain buffers or ingredients that function as buffers. For preparations to be buffered between pH 6 and 8, Sorensen's Phosphate Buffer is a useful system. It can be used for systemic, topical, or ophthalmic preparations. It has a relatively high buffer capacity.

Buffering agents may be selected accordingly, for example by employing HCl (pH 1-3), Citrate Buffer (pH 2.5-6.5), Acetate Buffer pH (3.6-5.6), Sorenson's Phosphate Buffer (pH 6-8), Sodium Bicarbonate (pH 8-9), Sodium Bicarbonate/Sodium Carbonate (pH 9-11), or NaOH (pH 11-13).

In order to raise the pH level of a glycolic acid solution, various approaches may be employed. For example, alkalizing agents may be used, for example selected from the group consisting of sodium hydroxide, ammonia solution, ammonium carbonate, diethanolamine, potassium hydroxide, sodium bicarbonate, sodium borate, sodium carbonate and trolamine.

Synergy:

To determine or quantify the degree of synergy or antagonism obtained by any given combination, a number of models may be employed. Typically, synergy is considered an effect of a magnitude beyond the sum of two known effects. In some embodiments, the combination response is compared against the expected combination response, under the assumption of non-interaction calculated using a reference model (refer Tang J. et al. (2015) What is synergy? The saariselkä agreement revisited. Front. Pharmacol., 6, 181).

Commonly utilized reference models include the Highest single agent (HSA) model (Berenbaum M. C. (1989) What is synergy. Pharmacol. Rev., 41, 93-141), the Loewe additivity model (Loewe S. (1953) The problem of synergism and antagonism of combined drugs. Arzneimiettel Forschung, 3, 286-290), the Bliss independence model (Bliss C. I. (1939) The toxicity of poisons applied jointly. Ann. Appl. Biol., 26, 585-615.), and more recently, the Zero interaction potency (ZIP) model (Yadav B. et al. (2015) Searching for drug synergy in complex dose-response landscapes using an interaction potency model. Comput. Struct. Biotechnol. J., 13, 504-505). The assumptions being made in these reference models are different from each other, which may produce somewhat inconsistent conclusions about the degree of synergy. Nevertheless, according to the present invention, when any one of these models indicates synergy between the agents in the combination as described herein, it may be assumed synergy has been achieved. Preferably, 2, 3 or all 4 of these models will reveal synergy between any two agents of the combination described herein.

Without limitation, four reference models are preferred, which can produce reliable results: (i) HSA model, where the synergy score quantifies the excess over the highest single drug response; (ii) Loewe model, where the synergy score quantifies the excess over the expected response if the two drugs are the same compound; (iii) Bliss model, where the expected response is a multiplicative effect as if the two drugs act independently; and (iv) ZIP model, where the expected response corresponds to an additive effect as if the two drugs do not affect the potency of each other.

The most widely used combination reference, and preferred model for determining synergy, is “Loewe additivity”, or the “Loewe model” (Loewe (1928), Ergebn. Physiol. 27:47-187; Loewe and Muischnek. “Effect of combinations: mathematical basis of the problem” Arch. Exp. Pathol. Pharmakol. 114: 313-326, 1926; Loewe S. (1953) The problem of synergism and antagonism of combined drugs. Arzneimittel Forschung, 3, 286-290), or “dose additivity” which describes the trade-off in potency between two agents when both sides of a dose matrix contain the same compound. For example, if 50% inhibition is achieved separately by 1 uM of drug A or 1 uM of drug B, a combination of 0.5 uM of A and 0.5 uM of B should also inhibit by 50%. Synergy over this level is especially important when justifying the clinical use of proposed combination therapies, as it defines the point at which the combination can provide additional benefit over simply increasing the dose of either agent.

As a further example of determining Loewe Additivity (or dose additivity), let d₁ and d₂ be doses of compounds 1 and 2 producing in combination an effect e. We denote by Dₑ₁ and Dₑ₂ the doses of compounds 1 and 2 required to produce effect e alone (assuming these conditions uniquely define them, i.e. that the individual dose-response functions are bijective). dₑ₁/Dₑ₂ quantifies the potency of compound 1 relatively to that of compound 2. d₂Dₑ₁/Dₑ₂ can be interpreted as the dose of compound 2 converted into the corresponding dose of compound 1 after accounting for difference in potency. Loewe additivity is defined as the situation where d₁+d₂Dₑ₁/Dₑ₂=Dₑ₁ or d₁/Dₑ₁+d₂/Dₑ₂₌₁. Geometrically, Loewe additivity is the situation where isoboles are segments joining the points (Dₑ₁, 0) and (0, Dₑ₂) in the domain (d₁, d₂). If we denote by f₁(d₁), f₂(d₂) and the dose-response functions of compound 1, compound 2 and of the mixture respectively, then dose additivity holds when d₁/f₁⁻¹ (f₁₂ (d₁, d₂))+d₂/f₂⁻¹ (f₁₂ d₂))=1.

Combined Administration:

According to the present invention, the term “combined administration”, otherwise known as co-administration or joint treatment, encompasses in some embodiments the administration of separate formulations of the compounds described herein, whereby treatment may occur within minutes of each other, in the same hour, on the same day, in the same week or in the same month as one another. Alternating administration of two agents is considered as one embodiment of combined administration. Staggered administration is encompassed by the term combined administration, whereby one agent may be administered, followed by the later administration of a second agent, optionally followed by administration of the first agent, again, and so forth. Simultaneous administration of multiple agents is considered as one embodiment of combined administration. Simultaneous administration encompasses in some embodiments, for example the taking of multiple compositions comprising the multiple agents at the same time, e.g. orally by ingesting separate tablets simultaneously. A combination medicament, such as a single formulation comprising multiple agents disclosed herein, and optionally additional medicaments, may also be used in order to co-administer the various components in a single administration or dosage.

A combined therapy or combined administration of one agent may precede or follow treatment with the other agent to be combined, by intervals ranging from minutes to weeks. In embodiments where the second agent and the first agent are administered separately, one would generally ensure that a significant period of time did not expire between the time of each delivery, such that the first and second agents would still be able to exert an advantageously combined synergistic effect on a treatment site. In such instances, it is contemplated that one would contact the subject with both modalities within about 12-24 h of each other and, more preferably, within about 6-12 h of each other, with a delay time of only about 12 h being most preferred. In some situations, it may be desirable to extend the time period for treatment significantly, however, where several days (2, 3, 4, 5, 6 or 7) to several weeks (1, 2, 3, 4, 5, 6, 7 or 8) lapse between the respective administrations.

In the meaning of the invention, any form of administration of the multiple agents described herein is encompassed by combined administration, such that a beneficial additional therapeutic effect, preferably a synergistic effect, is achieved through the combined administration of the two agents.

Treatment:

In the present invention “treatment” or “therapy” generally means to obtain a desired pharmacological effect and/or physiological effect. The effect may be prophylactic (preventative) in view of completely or partially preventing a disease and/or a symptom, for example by reducing the risk of a subject having a particular disease or symptom, or may be therapeutic in view of partially or completely curing a disease and/or adverse effect of the disease.

In the present invention, “therapy” includes arbitrary treatments of diseases or conditions in mammals, in particular, humans, for example, the following treatments (a) to (c): (a) Prevention of onset of a disease, condition or symptom in a patient; (b) Inhibition of a symptom of a condition, that is, prevention of progression of the symptom; (c) Amelioration of a symptom of a condition, that is, induction of regression of the disease or symptom.

Pharmaceutical Compositions and Methods of Administration:

The present invention also relates to a pharmaceutical composition comprising the compounds described herein. The invention also relates to pharmaceutically acceptable salts of the compounds described herein, in addition to enantiomers and/or tautomers of the compounds described.

The term “pharmaceutical composition” refers to a combination of the agent as described herein with a pharmaceutically acceptable carrier. The phrase “pharmaceutically-acceptable” refers to molecular entities and compositions that do not produce a severe allergic or similar untoward reaction when administered to a human. As used herein, “carrier” or “carrier substance” includes any and all solvents, dispersion media, vehicles, coatings, diluents, antibacterial and antifungal agents, isotonic and absorption delaying agents, buffers, carrier solutions, suspensions, colloids, and the like. The use of such media and agents for pharmaceutical active substances is well known in the art. Supplementary active ingredients can also be incorporated into the compositions.

The pharmaceutical composition containing the active ingredient may be in a form suitable for oral use, for example, as tablets, troches, lozenges, aqueous or oily suspensions, dispersible powders or granules, emulsions, hard or soft capsules, or syrups, solutions or elixirs. Compositions intended for oral use may be prepared according to any method known to the art for the manufacture of pharmaceutical compositions and such compositions. Tablets contain the active ingredient in admixture with non-toxic pharmaceutically acceptable excipients which are suitable for the manufacture of tablets. The tablets may be uncoated, or they may be coated by known techniques to delay disintegration and absorption in the gastrointestinal tract and thereby provide a sustained action over a longer period.

Dosage levels of the order of from about 0.01 mg to about 500 mg per kilogram of body weight per day are useful in the treatment of the indicated conditions. For example, a neurological condition may be effectively treated by the administration of from about 0.01 to 50 mg of the inventive molecule per kilogram of body weight per day (about 0.5 mg to about 5 g per patient per day). The amount of active ingredient that may be combined with the carrier materials to produce a single dosage form will vary depending upon the host treated and the particular mode of administration. For example, a formulation intended for the oral administration of humans may vary from about 5 to about 95% of the total composition. Dosage unit forms will generally contain between from about 1 mg to about 5000 mg of active ingredient. It will be understood, however, that the specific dose level for any particular patient will depend upon a variety of factors including the activity of the specific compound employed, the age, body weight, general health, sex, diet time of administration, route of administration, rate of excretion, drug combination and the severity of the particular disease undergoing therapy. The dosage effective amount of compounds according to the invention will vary depending upon factors including the particular compound, toxicity, and inhibitory activity, the condition treated, and whether the compound is administered alone or with other therapies.

The invention relates also to a process or a method for the treatment of the mentioned pathological conditions. The compounds of the present invention can be administered prophylactically or therapeutically, preferably in an amount that is effective against the mentioned disorders, to a warm-blooded animal, for example a human, requiring such treatment, the compounds preferably being used in the form of pharmaceutical compositions.

Administration/Injection/Intrathecal Administration:

As used herein, “administer” or “administration” refers to the delivery of the agent or combination of the present invention or a pharmaceutical composition thereof to an organism for the purpose of prevention or treatment of a disease. Suitable routes of administration may include, without limitation, oral, rectal, transmucosal or intestinal administration or intramuscular, subcutaneous, intramedullary, intrathecal, direct intraventricular, intravenous, intravitreal, intraperitoneal, intranasal, sublingual, buccal or intraocular injections.

A composition of the present invention may also be formulated for injection, e.g. parenteral administration, e.g., by bolus injection or continuous infusion. Formulations for injection may be presented in unit dosage form, e.g., in ampoules or in multi-dose containers, optionally with an added preservative. The compositions may take such forms as suspensions, solutions, or emulsions in oily or aqueous vehicles, and may contain formulating materials such as suspending, stabilizing, and/or dispersing agents.

Pharmaceutical compositions for parenteral administration, including intrathecal administration, include aqueous solutions of a water-soluble form of the active agent(s). Aqueous injection suspensions may contain substances that increase the viscosity of the suspension, such as sodium carboxymethyl cellulose, sorbitol, or dextran. Optionally, the suspension may also contain suitable stabilizers and/or agents that increase the solubility of the crystals of the present invention or a pharmaceutical composition thereof to allow for the preparation of highly concentrated solutions. Alternatively, the active ingredient may be in powder form for constitution with a suitable vehicle, e.g., sterile, pyrogen-free water, before use.

A preferred embodiment of the invention relates to intrathecal administration. Intrathecal administration is a route of administration for drugs via an injection into the spinal canal, or into the subarachnoid space so that it reaches the cerebrospinal fluid (CSF).

There are typically considered to be four methods of delivering medications intrathecally: two include the use of an external pump while the other two represent fully implantable devices. First, an external pump with a percutaneous catheter (tunneled or not tunneled) is less invasive to place and can be beneficial for patients. Second, for patients with a short life expectancy, totally implanted catheters with a subcutaneous injection port connected to an external pump may be more suitable. Third, a fully implanted fixed-rate (or constant flow) IDDS may be beneficial for long-term delivery of analgesia. Fixed-rate delivery systems are less expensive than variable-rate delivery systems and do not require a battery to operate, so should theoretically last the lifetime of the patient. The fourth method of spinal medication delivery consists of a fully implanted programmable IDDS, such as the Medtronic SynchroMed II infusion system (Medtronic Inc., Minneapolis, Minn., USA). These programmable devices deliver either an intermittent or continuous amount of medication intrathecally. Drug dosages can be changed without intervention such as the aspiration and refilling of a different medication concentration as seen in fixed-rate delivery systems.

Further embodiments relate to liquid formulations, and optionally transmucosal, preferably nasal, administration. As used herein, the term “transmucosal administration” refers to any administration of drug, pro-drug or active agent to a mucosal membrane. Transmucosal administration means are known in the art and relate preferably to oral, nasal, vaginal, and urethral modes. The transmucosal membranes are relatively permeable, have a rich blood flow and hence allow the rapid uptake of a drug into systemic circulation to avoid first pass metabolism. The oral transmucosal delivery preferably relate to the buccal and sublingual routes.

As used herein, the term “liquid” refers to its common meaning, including compositions with nearly incompressible fluid that conforms to the shape of its container but retains a (nearly) constant volume independent of pressure. As used herein “pharmaceutical compositions in liquid form” are liquids comprising one or more pharmaceutically active agents, suitable for administration to a subject, preferably a mammal, more preferably human subject. Liquid dosage forms are typically pharmaceutical products which involve a mixture of drug components and nondrug components (excipients). Liquid dosage forms are prepared: a) by dissolving the active drug substance in an aqueous or non-aqueous solvent (e.g. water, glycerin, ether, alcohol), or b) by suspending the drug in appropriate medium, or c) by incorporating the drug substance into an oil or water phase, such as suspensions, emulsions, syrups or elixirs.

Neurological Disease:

As used herein, the term “neurological disease” or disorder relates to any disorder of the nervous system. Structural, biochemical or electrical abnormalities in the brain, spinal cord or other nerves can result in a range of symptoms. Examples of symptoms include paralysis, muscle weakness, poor coordination, loss of sensation, seizures, confusion, pain, limitations in cognitive abilities and altered levels of consciousness. They may be assessed by neurological examination and studied and treated within the specialties of neurology and clinical neuropsychology.

In one embodiment, the neurological disease to be treated is selected from Alzheimer's and/or Parkinson's disease, dementia, schizophrenia, epilepsy, stroke, poliomyelitis, neuritis, myopathy, oxygen and nutrient deficiencies in the brain after hypoxia, anoxia, asphyxia, cardiac arrest, chronic fatigue syndrome, various types of poisoning, anaesthesia, particularly neuroleptic anaesthesia, spinal cord disorders, inflammation, particularly central inflammatory disorders, postoperative delirium and/or subsyndronal postoperative delirium, neuropathic pain, abuse of alcohol and drugs, addictive alcohol and nicotine craving, and/or effects of radiotherapy.

Neurodegenerative Disease:

The term “neurodegenerative diseases” is an umbrella term for diseases being associated with progressive loss of structure or function of neurons, including cell death of neurons. There are many parallels between different neurodegenerative disorders including atypical protein assemblies as well as induced cell death (in particular apoptosis). Neurodegenerative diseases affect many body activities, such as balance, movement, talking, breathing, and heart function. Many of these diseases are genetic. Sometimes the cause is a medical condition such as alcoholism, a tumor, or a stroke. Other causes may include toxins, chemicals, and viruses. The cause of some is, however, still not known. Neurodegenerative diseases are among the most serious health problems facing modern society. Many of these disorders become more common with advancing age, including Alzheimer's disease, Parkinson's disease, amyotrophic lateral sclerosis, and many others. The burden of these neurodegenerative diseases is growing inexorably as the population ages, with enormous economic and human costs.

All mentioned neurodegenerative diseases, i.e. Parkinson's disease, Alzheimer's disease, Huntington's disease, amyotrophic lateral sclerosis are known to be associated with a decline in mitochondrial activity (Lin and Beal (2006), Nature 443, 787-795). Means and methods for determining the mitochondrial activity are known in the art, for example from Agnello et al. (2008), Cytotechnology, 56(3):145-149.

Amyotrophic Lateral Sclerosis (ALS):

Amyotrophic lateral sclerosis (ALS), sometimes called Lou Gehrig's disease or classical motor neuron disease, is a rapidly progressive, invariably fatal neurological disease that attacks the nerve cells (neurons) responsible for controlling voluntary muscles. In ALS, both the upper motor neurons and the lower motor neurons degenerate or die, ceasing to send messages to muscles. Unable to function, the muscles gradually weaken, waste away, and twitch. Eventually the ability of the brain to start and control voluntary movement is lost. Symptoms are usually first noticed in the arms and hands, legs, or swallowing muscles. Muscle weakness and atrophy occur on both sides of the body. Individuals with ALS lose their strength and the ability to move their arms and legs, and to hold the body upright. Although the disease does not usually impair a person's mind or personality, several recent studies suggest that some people with ALS may develop cognitive problems involving word fluency, decision-making, and memory.

Parkinson's Disease:

One example of a neurodegenerative disease is Parkinson's disease. Parkinson's disease is caused by inexorable deterioration of dopaminergic neurons from the substantia nigra. Although little is known about the onset of Parkinson's disease, one clue is that a number of genes associated with the onset of Parkinson's disease are linked with mitochondrial activity. There is strong evidence that mitochondria' dysfunction and oxidative stress play a causal role in Parkinson's disease and in neurodegenerative disease pathogenesis in general. Other neurodegenerative diseases in which mitochondrial dysfunction and oxidative stress were observed include but are not limited to Alzheimer's disease, Huntington's disease, and amyotrophic lateral sclerosis (ALS) (Lin and Beal (2006), Nature 443, 787-795).

Alzheimer's Disease:

Alzheimer's disease (AD) is an age-related, non-reversible brain disorder that develops over a period of years. Initially, people experience memory loss and confusion, which may be mistaken for the kinds of memory changes that are sometimes associated with normal aging. However, the symptoms of AD gradually lead to behavior and personality changes, a decline in cognitive abilities such as decision-making and language skills, and problems recognizing family and friends. AD ultimately leads to a severe loss of mental function. These losses are related to the worsening breakdown of the connections between certain neurons in the brain and their eventual death. AD is one of a group of disorders called dementias that are characterized by cognitive and behavioral problems. It is the most common cause of dementia among people age 65 and older.

There are three major hallmarks in the brain that are associated with the disease processes of AD. (i) Amyloid plaques, which are made up of fragments of a protein called beta-amyloid peptide mixed with a collection of additional proteins, remnants of neurons, and bits and pieces of other nerve cells. (ii) Neurofibrillary tangles (NFTs), found inside neurons, are abnormal collections of a protein called tau. Normal tau is required for healthy neurons. However, in AD, tau clumps together. As a result, neurons fail to function normally and eventually die. (iii) Loss of connections between neurons responsible for memory and learning. Neurons cannot survive when they lose their connections to other neurons. As neurons die throughout the brain, the affected regions begin to atrophy, or shrink.

Huntington's Disease:

Huntington's disease (HD) results from genetically programmed degeneration of brain cells, called neurons, in certain areas of the brain. This degeneration causes uncontrolled movements, loss of intellectual faculties, and emotional disturbance. HD is a familial disease, passed from parent to child through a mutation in the normal gene. Each child of an HD parent has a 50-50 chance of inheriting the HD gene. If a child does not inherit the HD gene, he or she will not develop the disease and cannot pass it to subsequent generations. A person who inherits the HD gene will sooner or later develop the disease. Whether one child inherits the gene has no bearing on whether others will or will not inherit the gene. Some early symptoms of HD are mood swings, depression, irritability or trouble driving, learning new things, remembering a fact, or making a decision. As the disease progresses, concentration on intellectual tasks becomes increasingly difficult and the patient may have difficulty feeding himself or herself and swallowing. The rate of disease progression and the age of onset vary from person to person. A genetic test, coupled with a complete medical history and neurological and laboratory tests, helps physicians diagnose HD.

Stimulating Neuronal Plasticity, Enhancing Psychotherapy and Schizophrenia Treatment:

Psychotherapy is a key therapeutic tool for treating mental disorders. The earliest recorded approaches were a combination of religious, magical and/or medical perspectives. It wasn't until the end of the 19th century, around the time when Sigmund Freud was first developing his “talking cure” in Vienna, that the first scientifically clinical application of psychology began. Since then different types of psychotherapy have been developed (e.g. psychoanalysis, cognitive behavioural therapy, behaviour therapy, group therapy, expressive therapy, narrative therapy or gestalt therapy) and are used in the clinical setting. The type of psychotherapy used depends on the underlying disorder and the need of the patient.

It has been shown that psychiatric disorders alter the normal activity patterns of certain brain regions in a disease specific manner: Obsessive-compulsive disorder (OCD) has been associated with hypermetabolism in the orbitofrontal cortex, the anterior cingulate gyrus and the head of the caudate nucleus. Panic disorder has been traditionally associated with neurofunctional alterations in the ‘fear network’, involving both limbic and cortical structures Functional neuroimaging studies of patients with major depression have consistently reported reduced metabolism in frontal and temporal regions, the insula and the basal ganglia. These studies have also provided preliminary evidence that hippocampal metabolism is associated with severity of depression. Posttraumatic stress disorder (PTSD) seems to be linked to increased amygdala activation by trauma-related stimuli and trauma unrelated emotional material. Another widely reported finding is decreased activation in medial prefrontal cortex in relation to script-driven imagery, trauma-related, and -unrelated, emotional, and neutral stimuli. Schizophrenia has been associated with regional alterations in a distributed network that includes the dorsolateral prefrontal cortex, the anterior cingulate cortex and both lateral and medial temporal regions.

Psychotherapy uses neural plasticity to revert the effects of psychiatric disorders on the activity patterns of the brain. Psychotherapy can have a profound influence on a person's belief system, emotional state and behaviour. Psychotherapy, alone or in combination with psychotropic drugs, can revert these changes and have a profound impact on the activity patterns in unrelated brain regions. All psychotherapy-induced changes require re-wiring of the neuronal networks implicated, changes in the way neurons connect within given neuronal circuits and their reaction to external cues. In summary, all these changes are based on an impressive characteristic of neurons, neural plasticity.

As used herein, neuroplasticity (or neural plasticity) refers to the ability of neurons to change in form and function in response to alterations in their environment. Neurons function as parts of local circuits in the brain, and each neuron can change its functional role in a circuit by altering how it responds to inputs or influences other neurons. Variations in neuroplasticity are development-dependent and region specific. It peaks at different time-points after conception and in certain regions to facilitate acquiring certain abilities (e.g. early increases in primary and secondary sensori-motor brain areas to facilitate the acquisition of primary sensori-motor functions).

Age-related reduction in neuroplasticity has been associated with certain alterations in neurons, including:
Small, region-specific changes in dendritic branching and spine density.
Reduction in neuronal number in certain areas of the brain
Increase in Ca²⁺ conductance in aged neurons.
Ca2+ activates outward K+ currents that are responsible for the afterhyperpolarizing potential (AHP) that follows a burst of action potentials. Aged neurons in areas CA1 and CA3 have an increase in the amplitude of the AHP that results, at least in part, from age-related increases in Ca2+ conductance. The larger AHP observed in aged hippocampal neurons suggests that aged CA1 pyramidal cells are less excitable, as they are further from action potential threshold than are young neurons during the AHP.
Reduced synapse number (up to 30% reduction). This reduction is accompanied by a decrease in the presynaptic fibre potential amplitude.
Age related changes in gene expression. The behaviourally relevant up-regulated genes included several that are associated with inflammation and intracellular Ca2+ release pathways, whereas genes associated with energy metabolism, biosynthesis and activity-regulated synaptogenesis were down-regulated (e.g. c-fos).

The effects of altered morphology, changes in gene expression, biophysical properties and synaptic connections of aged neurons on plasticity can be assessed by measuring age-associated alterations in long-term potentiation (LTP) and long-term depression (LTD). LTP can be divided into an induction phase (early-phase LTP) and a maintenance phase (late-phase LTP). The induction phase involves the temporal association of presynaptic glutamate release with postsynaptic depolarization (necessary to eject Mg2+ from the pores of NMDA (N-methyl-d-aspartate) receptors), which results in an increase in intracellular Ca2+. LTP maintenance is the continued expression of increased synaptic efficacy that persists after induction. It probably involves changes in gene expression and insertion of AMPA receptors into the postsynaptic membrane. Aged rats have deficits in both LTP induction and maintenance.

In the case of schizophrenia, it is thought that pre- and postnatal alterations in neuronal migration of different types of neurons and postnatal problems in myelination lead to alterations in the connectivity between neurons thereby dramatically reducing neuroplasticity. This is thought to lead to the characteristic drop (knick) in the curve of both high-cognitive and socio-affective functions observed in schizophrenic patients.

Substances and non-pharmacological approaches able to reverse the above-mentioned alterations and enhance neuroplasticity could exponentially increase the therapeutic effect of psychotherapy in adult patients and improve cognitive and socio-affective functions in schizophrenic patients.

Regarding neuroplasticity enhancing substances, several studies have shown the potential of ketamine (and es-ketamin) and other rapid acting antidepressants including NMDA channel blockers, glycine site agents, and allosteric modulators in neural plasticity. Also, the hematopoietic growth factor erythropoetin (EPO), involved in brain development, has been associated with the production and differentiation of neuronal precursor cells thereby enhancing neuroplasticity. It has also been shown that Ketamine, a N-methyl-D-aspartate (NMDA) receptor antagonist that produces rapid and sustained antidepressant actions even in treatment-resistant patient, enhances structural plasticity in mouse mesencephalic neurons and human iPSC-derived dopaminergic neurons.

Based on these findings, the present invention further relates to the use of GA (preferably in the combination as described herein) for stimulating neuroplasticity, and thereby treating or enhancing the treatment, for example by psychotherapy or other therapeutic approaches, of diseases or conditions that would benefit from enhanced neural plasticity. For example, psychiatric disorders, such as obsessive-compulsive disorder (OCD), panic disorder, depression, posttraumatic stress disorder (PTSD) and schizophrenia may be treated or the treatment of these conditions may be enhanced using GA, preferably in the combination of the invention.

Stimulating Mitochondrial Function and ATP Production:

As used herein, the term “mitochondria function”, otherwise referred to as “mitochondrial metabolism”, relates to the process of mitochondria respiration (oxidative phosphorylation). Mitochondria have a central role in energy metabolism. Part of the free energy derived from the oxidation of food is transformed inside mitochondria to ATP, which depends on oxygen. When oxygen is limited, glycolytic products are metabolized directly in the cytosol by the less efficient anaerobic respiration that is independent of mitochondria. The mitochondrial ATP production relies on the electron transport chain (ETC), composed of respiratory chain complexes I-IV, which transfer electrons in a stepwise fashion until they finally reduce oxygen to form water. The NADH and FADH2 formed in glycolysis, fatty-acid oxidation and the citric acid cycle are energy-rich molecules that donate electrons to the ETC. Electrons move toward compounds with more positive oxidative potentials and the incremental release of energy during the electron transfer is used to pump protons (H+) into the intramembrane space. Complexes I, III and IV function as H+ pumps that are driven by the free energy of coupled oxidation reactions. During the electron transfer, protons are always pumped from the mitochondrial matrix to the intermembrane space, resulting in a potential of ˜150-180 mV. The proton gradient generates a chemiosmotic potential, also known as the proton motive force, which drives the ADP phosphorylation via the ATP synthase (FoF1 ATPase—complex V). The Fo domain of ATPase couples a proton translocation across the inner mitochondrial membrane with the phosphorylation of ADP to ATP. The rate of mitochondrial respiration depends on the phosphorylation potential expressed as a [ATP]/[ADP] [Pi] ratio across the inner mitochondrial membrane that is regulated by the adenine nucleotide translocase (ANT).

As used herein, an increase in mitochondrial metabolism and an increased mitochondrial function in particular refer to an increased rate of mitochondrial respiration/oxidative phosphorylation.

Mitochondrial metabolism is an indicator of mitochondrial function and can be analyzed for example by measuring the rate of oxidative phosphorylation, the mitochondrial membrane potential (MtMP), cellular levels of reactive oxygen species (ROS), wherein an increased rate of oxidative phosphorylation, a high mitochondrial membrane potential (MtMP), and low levels of reactive oxygen species (ROS) are indicative of functional mitochondria and a high or intact mitochondrial metabolism. Also, NADH and NADPH levels can be determined as an indicator of mitochondrial function and metabolism, wherein high levels are indicative of good functionality. Further indicators of mitochondrial functionality and metabolism are expression levels of genes that are centrally involved in mitochondrial function and biogenesis, which include nuclear and mitochondrial genes, such as Nrf1, Tfam, Nd1, Cytb, Co1 and Atp6, among others known to the skilled person. In contrast, a (concomitant) upregulation of glycolytic enzymes can be indicative of a declining mitochondrial metabolism. Furthermore, high ATP levels are an indicator of intact mitochondrial function and mitochondrial metabolism. A declined of mitochondrial function can be observed by determining the parameters above and comparing them to a previously determined value or other reference values.

If mitochondrial function increases, it means that mitochondrial metabolism becomes more active and more efficient. This leads to an increase in ATP production. Through this pathway, several physiological functions that decrease during aging can be restored and lead to age-related diseases. Among diverse factors that contribute to human aging, the mitochondrial dysfunction has emerged as one of the key hallmarks of aging process and is linked to the development of numerous age-related pathologies including metabolic syndrome, neurodegenerative disorders, cardiovascular diseases and cancer. Mitochondria are central in the regulation of energy and metabolic homeostasis, and harbor a complex quality control system that limits mitochondrial damage to ensure mitochondrial integrity and function (reviewed in The Mitochondrial Basis of Aging and Age-Related Disorders Sarika Srivastava, Genes, 2017.

Ischemic Disease:

The terms “ischemic insult”, “ischemic disease” or “ischemic disorder” are used interchangeably herein, and designate the acute or sub-acute interruption of the blood supply to one or more bodily tissues. As discussed herein, ischemic insults are commonly due to the occlusion of an artery, either by: i) arteriosclerosis, ii) the rupture of an arteriosclerotic plaque or an aneurisma with or without the in situ formation of a clot, iii) the rupture of an artery causing an haemorrhage or iv) an embolic event in which a clot (arterio-arterial or veno-arterial embolism), an air bubble (gaseous embolism) or lipid tissue (lipid embolism) formed elsewhere is transported in the blood until it occludes an artery with a smaller diameter.

In one embodiment the invention relates to the treatment of brain global ischemia. Brain global ischemia is a particular condition in which there is insufficient blood flow to the brain to meet metabolic demand. This leads to poor oxygen supply or cerebral hypoxia and thus to the death of brain tissue or cerebral infarction/ischemic stroke. This general reduction of blood supply to the brain is normally due to a heart failure or a dramatic drop in the blood pressure. The main parameters influencing the functional outcome of an ischemic event are the cellular death rate and the size of ischemic tissue, both aspects of the disease being interrelated with one another.

In particular embodiments of the invention ischemic disease to be treated and/or prevented may be (a) cerebral ischemia, in particular stroke and subarachnoid hemorrhage, vascular dementia and/or infarct dementia; (b) myocardial ischemia, in particular a coronary heart disease and/or myocardial infarction; (c) peripheral limb disease, in particular periphery arterial occlusive disease, (d) renal and/or intestinal ischemia, in particular intestinal infarction due to the occlusion of the celiac or mesenteric arteries.

With respect to the prevention of ischemic disease in a patient at risk thereof, the patient at thereof may demonstrate one or more of the following indications: (a) shows symptoms or indications of being at risk of developing a ischemic disease, such as high blood cholesterol and triglyceride levels, high blood pressure (wherein references to “high” levels refer to levels above the average population values), the presence of diabetes and prediabetes, overweight, tobacco smoking, lack of physical activity, an unhealthy diet and/or stress; (b) shows any risk markers in ex vivo tests, in particular in blood samples; (c) has previously suffered from an ischemic disease, in particular had a cerebral or myocardial ischemia; and/or (d) has a predisposition of developing a cardiovascular ischemic disease, in particular a genetic predisposition.

Stroke:

A stroke is a medical condition in which poor blood flow to the brain causes cell death. There are two main types of stroke: ischemic, due to lack of blood flow, and haemorrhagic, due to bleeding. Both cause parts of the brain to stop functioning properly. Signs and symptoms of a stroke may include an inability to move or feel on one side of the body, problems understanding or speaking, dizziness, or loss of vision to one side. Signs and symptoms often appear soon after the stroke has occurred.

Male Infertility/Sperm Motility:

The term “infertility” designates the inability of an animal to conceive sexual offspring. The term “male infertility” refers to a male's inability to cause pregnancy in a fertile female. Male infertility is commonly due to deficiencies in the semen (spermatozoa), and the assessment of semen quality is used in the art as a surrogate to measure of male fertility. The male infertility is in accordance with the invention the male infertility of a mammal.

Semen deficiencies which cause male infertility may be labelled as follows: (i) Oligospermia or oligozoospermia—decreased number of spermatozoa in semen; (ii) aspermia—complete lack of semen; (iii) hypospermia—reduced seminal volume; (iv) azoospermia—absence of sperm 15 cells in semen; (v) teratospermia—increase in sperm with abnormal morphology, and (vi) asthenozoospermia—reduced sperm motility/mobility. There are various combinations of these deficiencies as well, e.g. Teratoasthenozoospermia, which is reduced sperm morphology and motility. Moreover, low sperm counts are often associated with decreased sperm motility and increased abnormal morphology, thus the terms “oligoasthenoteratozoospermia” or “oligospermia” can be used as a catch all these deficiencies.

The two aspects typically analyzed in order to diagnose a lack of sperm motility are in general: the percentage of sperm cells moving within the semen sample, and a count of the total number of moving sperm. Sperm progressivity is determined by the ability of the sperm to swim forward, thus allowing the sperm to follow a concentration gradient of signalling molecules in the vagina and uterus that guide the sperm to reach the egg in order for fertilization to happen. Progressive motility means the sperm is active, whether moving linearly. In nonprogressive motility, the sperm is active although there is no forward progression. When sperm does not move, this is referred to as immotility/immobility.

Anti-Ageing Applications:

In embodiments of the invention, the pharmaceutical combination may be used for the treatment and/or prevention of an age-related medical condition associated with a decline in mitochondrial function, wherein said treatment and/or prevention comprises slowing, reversing and/or inhibiting the ageing process

In embodiments of the invention, the age-related medical condition is an aging-associated disease. In further embodiments, the age-related medical condition is an aging-associated dysfunction. In embodiments of the invention, the age-related medical condition, which may be an aging-associated disease or dysfunction, is associated with a decline in mitochondrial function.

In embodiments, the age-related medical condition associated with a decline in mitochondrial function is selected from the group comprising or consisting of myocardial dysfunction, myocardial infarction, heart failure, liver failure, nonalcoholic fatty liver disease (NAFLD), nonalcoholic steatohepatitis (NASH), chronic kidney disease, acute kidney injury, kidney failure, muscle atrophy, sarcopenia, cardiomyopathy, cardiovascular disease, cancer, diabetes, metabolic syndrome, neuropathies, neurodegenerative disorders such as amyotrophic lateral sclerosis (ALS), multiple sclerosis, Parkinson's disease, and Alzheimer's disease.

In embodiments, the treatment and/or prevention of an age-related medical condition comprises slowing, reversing and/or inhibiting the ageing process.

As used in the context of the present invention, the term age-related medical condition comprises aging-associated diseases, aging-associated dysfunctions, such as aging-associated organ dysfunctions, and conditions associated with a decline in mitochondrial function.

Age-related medical conditions are changes in the health status of a subject that occur with age due to changes in organ and cell functions that depend on the age of the subject. During aging the incidence of acute and chronic conditions such as neurological disorders, diabetes, degenerative arthritis, and cancer rises within individuals, so that aging has been termed the substrate on which age-associated diseases grow. The invention therefore relates to prophylactic and symptomatic treatment of diseases associated with ageing.

The molecular pathways underlying aging are only partially understood, as large individual heterogeneity of the biological aging process is observed. These inter-individual differences are proposed to derive from accumulation of stochastic damage that is counteracted by genetically encoded and environmentally regulated repair systems. Aging associated mitochondrial dysfunction by itself is thought to contribute to stem cell and tissue aging. The present invention therefore provides means for the treatment and/or prevention and/or reduction in risk of ageing as such, in addition to age-related medical conditions.

As used herein, an aging associated disease is a disease that is most often seen with increasing frequency with increasing age of the subject or patient. Essentially, aging-associated diseases are complications arising from aging or senescence. “Aging-associated disease” is used here to mean “diseases of the elderly”, so diseases incurring with higher frequency in older individuals. Non-exhaustive examples of aging-associated diseases are atherosclerosis and cardiovascular disease, cancer, arthritis, cataracts, osteoporosis, type 2 diabetes, hypertension and neurodegenerative diseases, such as Alzheimer's disease. The incidence of such aging associated diseases increases exponentially with age.

Aging associated diseases of the invention comprise in particular circulatory disorders, cardiovascular disease, artery or blood vessel conditions and/or ischemic obstructive or occlusive diseases or conditions refer to states of vascular tissue where blood flow is, or can become, impaired or altered from normal levels. Many pathological conditions can lead to vascular diseases that are associated with alterations in the normal vascular condition of the affected tissues and/or systems. Examples of vascular conditions or vascular diseases to which the methods of the invention apply are those in which the vasculature of the affected tissue or system is senescent or otherwise altered in some way such that blood flow to the tissue or system is reduced or in danger of being reduced or increased above normal levels. It refers to any disorder in any of the various parts of the cardiovascular system, which consists of the heart and all of the blood vessels found throughout the body.

Neurodegenerative disease or neurodegeneration is a term for aging associated medical conditions in which the progressive loss of structure or function of neurons, including death of neurons, occurs. Many neurodegenerative diseases, including ALS, Parkinson's, Alzheimer's, and Huntington's, occur as a result of neurodegenerative processes. Such diseases are commonly considered to be incurable, resulting in progressive degeneration and/or death of neuron cells. A number of similarities are present in the features of these diseases, linking these diseases on a sub-cellular level. Some of the parallels between different neurodegenerative disorders include atypical protein assembly as well as induced cell death. Dementia is a group of brain diseases causing a gradual decline of cognitive functions. Most of these diseases are chronic neurodegenerative diseases and are associated with neurobehavioral and/or neuropsychiatric symptoms that disable patients to independently perform activities of daily live.

In some embodiments, the treatment and/or prevention of an age-related medical condition associated with a decline in mitochondrial function, wherein said treatment and/or prevention comprises slowing, reversing and/or inhibiting the ageing process, does not include neurodegenerative disease.

In some embodiments, the treatment and/or prevention of an age-related medical condition associated with a decline in mitochondrial function, wherein said treatment and/or prevention comprises slowing, reversing and/or inhibiting the ageing process, does not include ischemic, cardiovascular or circulatory disease.

Aging associated diseases comprise diabetes mellitus, which is a group of chronic metabolic diseases that are associated with high blood sugar levels over prolonged periods, which can lead to severe complications including cardiovascular diseases, stroke, kidney failure, foot ulcers and damaged eyes. The two main subtypes are type 1 and type 2 diabetes mellitus. Type 1 diabetes mellitus is characterized by the loss of insulin-producing cells in the pancreas. It accounts for about 10% of the diabetes cases in the US and Europe, mostly affects children and is often associated with autoimmune pathologies. Type 2 diabetes mellitus is characterized by insulin resistance. Diabetes mellitus represents a massive health issue with more than 350 million affected people in 2013 worldwide. Diabetes mellitus according to the present invention refers to, but is not limited to, one or more of, type 1 diabetes mellitus, type 2 diabetes mellitus, gestational diabetes, and latent autoimmune diabetes of adults.

Metabolic syndrome is another example of an aging associated disease of the invention. Metabolic syndrome is a clustering of at least three of the five following medical conditions: central obesity, high blood pressure, high blood sugar, high serum triglycerides, and low serum high-density lipoprotein (HDL). Metabolic syndrome is associated with the risk of developing cardiovascular disease and type 2 diabetes. The syndrome is thought to be caused by an underlying disorder of energy utilization and storage, including dysfunction of mitochondrial metabolism. The continuous provision of energy via dietary carbohydrate, lipid, and protein fuels, unmatched by physical activity/energy demand creates a backlog of the products of mitochondrial oxidation, a process associated with progressive mitochondrial dysfunction and insulin resistance.

Further aging associated disease of the invention comprise disease of the liver and the kidney, such as liver failure, nonalcoholic fatty liver disease (NAFLD), nonalcoholic steatohepatitis (NASH), chronic kidney disease, acute kidney injury, kidney failure.

Aging associated diseases also comprise neuropathy, often also referred to as peripheral neuropathy. Neuropathy is a disease affecting the peripheral nerves, meaning nerves beyond the brain and spinal cord. Damage to peripheral nerves may impair sensation, movement, gland or organ function depending on which nerves are affected; in other words, neuropathy affecting motor, sensory, or autonomic nerves result in different symptoms. More than one type of nerve may be affected simultaneously. Peripheral neuropathy may be acute (with sudden onset, rapid progress) or chronic (symptoms begin subtly and progress slowly), and may be reversible or permanent.

Muscle atrophy is another aging associated disease of the invention. It is characterized by the loss of skeletal muscle mass that can be caused by immobility, aging, malnutrition, medications, or a wide range of injuries or diseases that impact the musculoskeletal or nervous system. Sarcopenia is the muscle atrophy associated with aging and can be slowed by exercise. Finally, diseases of the muscles such as muscular dystrophy or myopathies can cause atrophy, as well as damage to the nervous system such as in spinal cord injury or stroke. Muscle atrophy results from an imbalance between protein synthesis and protein degradation, although the mechanisms are incompletely understood and are variable depending on the cause. Muscle loss can be quantified with advanced imaging studies, but this is not frequently pursued.

Sarcopenia is an aging associated disease of the invention characterized by the degenerative loss of skeletal muscle mass, quality, and strength associated with aging and immobility. The rate of muscle loss is dependent on exercise level, co-morbidities, nutrition and other factors. Sarcopenia can lead to reduction in functional status and cause disability. The muscle loss is related to changes in muscle synthesis signaling pathways. It is distinct from cachexia, in which muscle is degraded through cytokine-mediated degradation, although both conditions may co-exist. Sarcopenia is considered a component of the frailty syndrome. Changes in hormones, immobility, age-related muscle changes, nutrition and neurodegenerative changes have all been recognized as potential causative factors.

Cancer is an age-related disease. The term “cancer” comprises a group of diseases that can affect any part of the body and is caused by abnormal cell growth and proliferation. These proliferating cells have the potential to invade the surrounding tissue and/or to spread to other parts of the body where they form metastasis. The incidence of cancer in increasing with age and cancer is therefore considered an aging associated disease of the present invention. Cancer according to the present invention refers to all types of cancer or neoplasm or malignant tumors found in mammals, including leukemias, sarcomas, melanomas and carcinomas. Examples of cancers are cancer of the breast, pancreas, colon, lung, non-small cell lung, ovary, and prostate.

Additional cancers include, but are not limited to Hodgkin's Disease, Non-Hodgkin's Lymphoma, multiple myeloma, neuroblastoma, breast cancer, ovarian cancer, lung cancer, rhabdomyosarcoma, primary thrombocytosis, primary macroglobulinemia, small-cell lung tumors, primary brain tumors, stomach cancer, colon cancer, malignant pancreatic insulanoma, malignant carcinoid, urinary bladder cancer, premalignant skin lesions, testicular cancer, lymphomas, thyroid cancer, esophageal cancer, genitourinary tract cancer, malignant hypercalcemia, cervical cancer, endometrial cancer, adrenal cortical cancer, and prostate cancer.

In embodiments, the age-related condition is an aging associated dysfunction of cellular functions, such as a dysfunction of mitochondrial metabolism or other cellular mechanisms that lead to cellular and ultimately organ dysfunction leading to a clinical manifestation, such as an aging associated disease. Many aging associated diseases are also associated with a decline in mitochondrial function. This group comprises in particular myocardial dysfunction, myocardial infarction, heart failure, liver failure, nonalcoholic fatty liver disease (NAFLD), nonalcoholic steatohepatitis (NASH), chronic kidney disease, acute kidney injury, kidney failure, muscle atrophy, sarcopenia, cardiomyopathy, cardiovascular disease, cancer, diabetes, metabolic syndrome, neuropathies, neurodegenerative disorders such as amyotrophic lateral sclerosis (ALS), multiple sclerosis, Parkinson's disease, and Alzheimer's disease.

In some preferred embodiments, the invention seeks to provide an anti-ageing effect, or otherwise termed as the slowing, reversing and/or inhibiting the ageing process. In some embodiments, the prophylactic effect or reduced occurrence or severity of age-related disease or symptoms thereof will occur. In some embodiments, increased lifespan as such will occur, due to the slowing of the ageing process, induced by the enhanced ATP production and mitochondrial function stimulated by the GA treatment, or treatment with the inventive combination.

Immune Stimulation/Enhancement:

Mitochondria are well appreciated for their role as biosynthetic and bioenergetic organelles. In the past two decades, mitochondria have emerged as signaling organelles that contribute critical decisions about cell proliferation, death and differentiation. Mitochondria not only sustain immune cell phenotypes but also are necessary for establishing immune cell phenotype and their function. Mitochondria can rapidly switch from primarily being catabolic organelles generating ATP to anabolic organelles that generate both ATP and building blocks for macromolecule synthesis. This enables them to fulfill appropriate metabolic demands of different immune cells (reviewed in Immunity. 2015 Mar. 17; 42(3): 406-417).

Various examples are known regarding mitochondrial function and regulation of the immune system. For example, mitochondrial signaling dictates macrophage polarization and function, and mitochondrial signaling is necessary for responses to activators of innate immune signaling. Mitochondrial signaling also controls adaptive immunity and regulates CD8+ memory T cell formation. Through the stimulation of mitochondrial function by treatment with GA, or the combination of the invention, the immune system can be stimulated accordingly and provide an enhanced therapeutic benefit to a subject in need of immune stimulation.

For example, it has been shown that that T cells with dysfunctional mitochondria act as accelerators of senescence. In mice, these cells instigate multiple aging-related features, including metabolic, cognitive, physical, and cardiovascular alterations, which together result in premature death. T cell metabolic failure induces the accumulation of circulating cytokines, which resembles the chronic inflammation that is characteristic of aging (“inflammaging”). This cytokine storm itself acts as a systemic inducer of senescence (Desdín-Micó et al. Science, 2020).

Immune Regulation:

Calcium homeostasis and calcium signaling are well appreciated for their numerous functions in the body. Calcium is essential for inter- and intracellular signaling in all cell types. Excesses in calcium lead to the activation of apoptosis and cell death (e.g. during ischemia). Calcium flux across the membrane and its downstream signaling regulates several cellular functions like exocytosis, protein production in the ER, mitochondrial morphology and function through the regulation of energy production (calcium is essential for the Kreb's cycle), intracellular transport (including axonal/neurite transport) and many other cellular processes. Interestingly, it also plays an important role in the reaction of the immune system to external effectors. The regulation of calcium homeostasis through GA could be beneficial to obtain a proper reaction of the immune system. Several studies have shown that in cells of the immune system, calcium signals are essential for diverse cellular functions including differentiation, effector function and gene transcription through storage operated calcium entry. After engagement of immunoreceptors such as T-cell and B-cell antigen receptors and the Fc receptors on mast cells and NK cells, “store-operated” Ca2+ entry constitutes the major pathway of intracellular Ca2+ increase (reviewed in “Calcium signaling in lymphocytes” Masatsugu Oh-hora and Anjana Rao, Current Opinion in Immunology 2008, https://www.ncbi.nlm.nih.gov/pmc/articles/PMC2574011/)

Embryonic Development and Oocyte Reproductive Fitness:

It has been shown that fertility in woman decrease during aging. Maternal age is the main cause of embryonic aneuploidies. More than 90% of these imbalances are indeed of maternal origin caused by chromosomal missegregation during oogenesis and meiosis (a special form of mitosis). Mainly meiosis I errors may occur (>70% of cases). Meiosis and mitosis play therefore an essential role in fertilization and embryonic development, in which cell division occurs at a high rate and with great precision.

Also mitochondria and their correct function play a key role in fertilization and embryonic development. Mitochondria are the most numerous organelles in the oocyte and represent its powerhouse. They are characterized by their own genome (mtDNA) and constitute the main maternal contribution to embryogenesis. Indeed, the sperm does not provide mitochondria to the offspring. They are considered pivotal especially in the delicate first phases of preimplantation development, when a balanced energy consumption is crucial for an efficient oocyte cytoplasmic and nuclear maturation, throughout processes such as germinal vesicle breakdown, or microtubule assembly and disassembly during meiotic spindle formation. Moreover, mitochondria cover an essential role in various signaling pathways, such as Ca2+ signaling and regulation of the intracellular red-ox potential, particularly important for fertilization and early development. The adverse effect of aging upon the mitochondria within the oocyte has been widely reported: mitochondrial swelling, vacuolization, and cristae alteration have been described as common structural features of oocytes from AMA patients. For instance, a reduced ATP production and decreased metabolic activity in aged oocytes has been highlighted, which in turn may contribute to impairments in meiotic spindle assembly, cell cycle regulation, chromosome segregation, embryo development, and finally implantation. Early Ovarian Ageing is a medical condition that is associated with a premature aging of the oocytes in woman already in the early 30s.

In some preferred embodiments, the invention seeks to provide a positive effect on fertility fitness, or otherwise termed as the slowing, reversing and/or inhibiting the ageing process of the oocytes. In some embodiments, the prophylactic effect or reduced occurrence or severity of oocyte fitness.

### FIGURES

The invention is further described by the figures. These are not intended to limit the scope of the invention.

### SHORT DESCRIPTION OF THE FIGURES

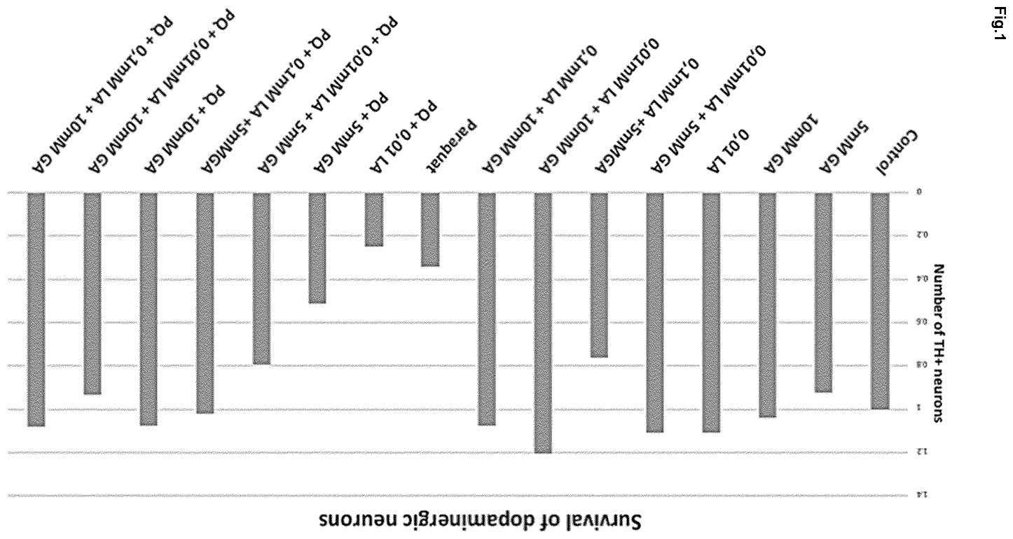

FIG. 1: GA combined with LA is more effective than GA alone in protecting the toxic effect of Paraquat on dopaminergic neurons.

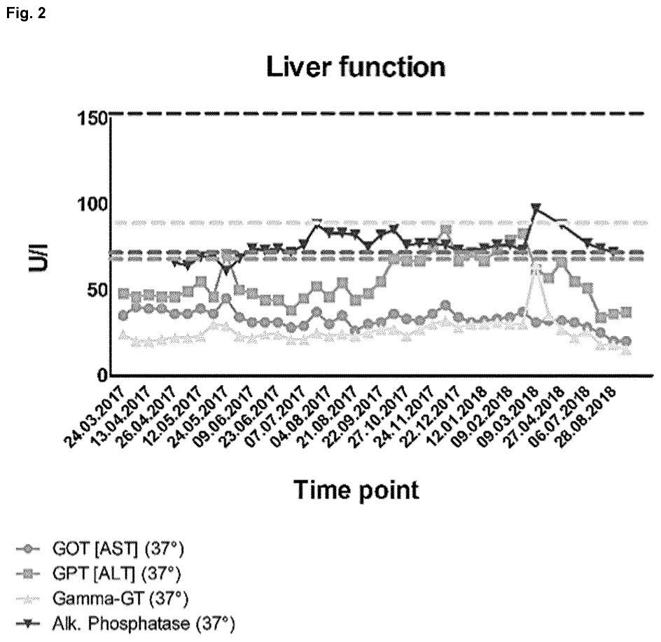

FIG. 2: Liver Function: Individualised clinical trial data from a FUS patient with ALS.

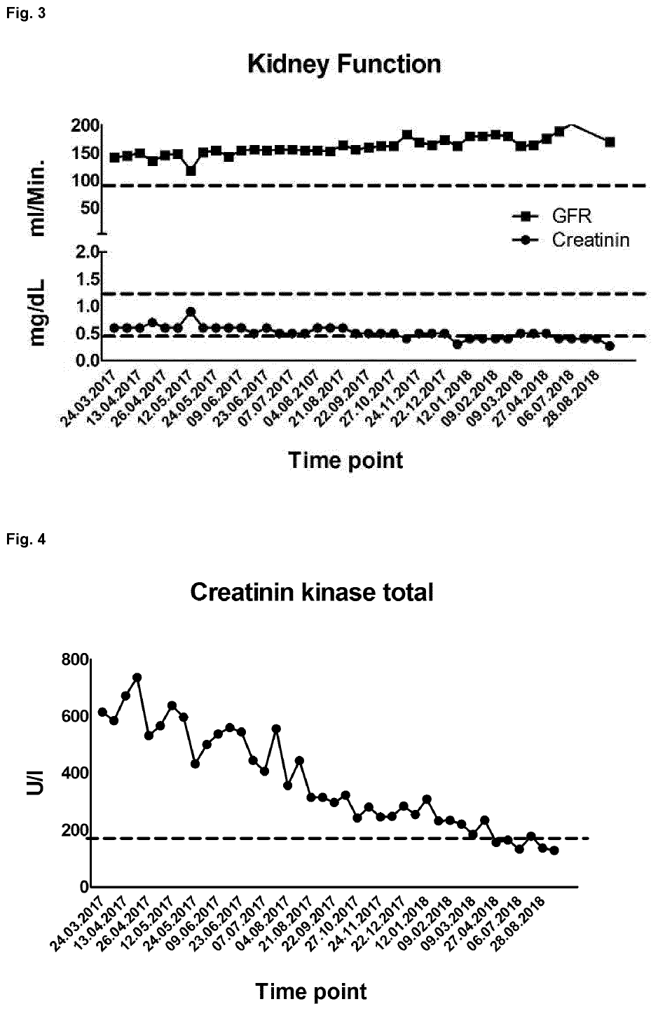

FIG. 3: Kidney function: Individualised clinical trial data from a FUS patient with ALS.

FIG. 4: Creatine Kinase: Individualised clinical trial data from a FUS patient with ALS.

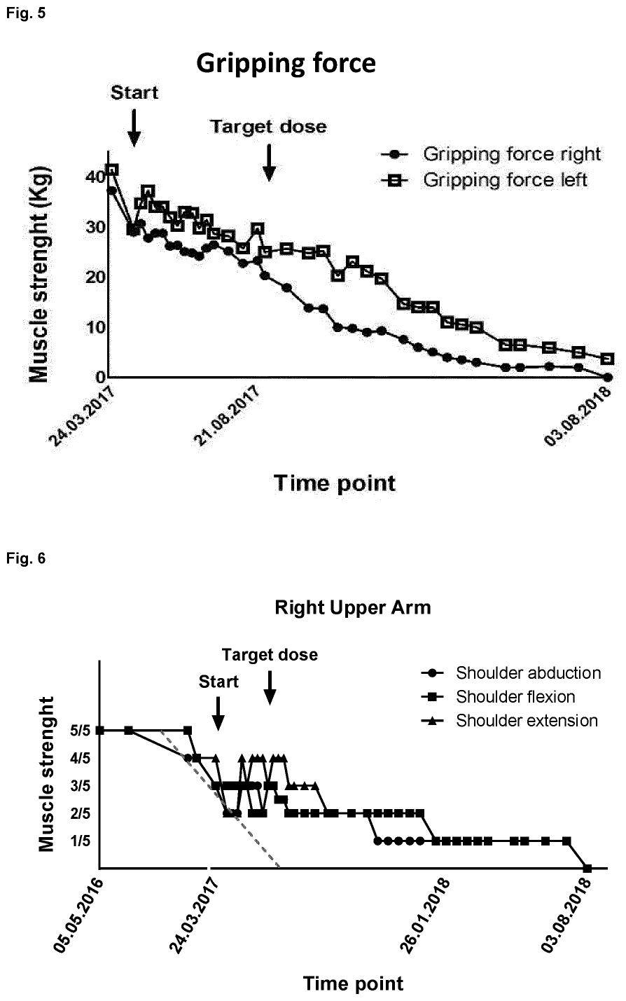

FIG. 5: Gripping Force: Individualised clinical trial data from a FUS patient with ALS.

FIG. 6: Muscle Strength Arm: Individualised clinical trial data from a FUS patient with ALS.

FIG. 7: Muscle Strength Leg: Individualised clinical trial data from a FUS patient with ALS.

FIG. 8: Pharmacokinetics: Blood concentration of GA after administration.

FIG. 9: CSF concentration of GA after administration.

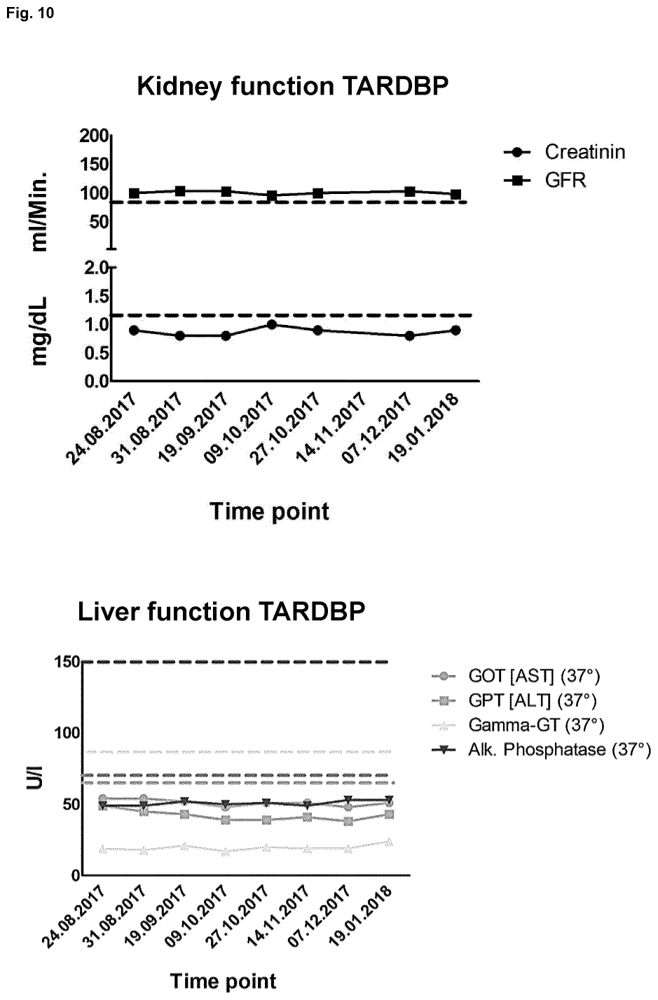

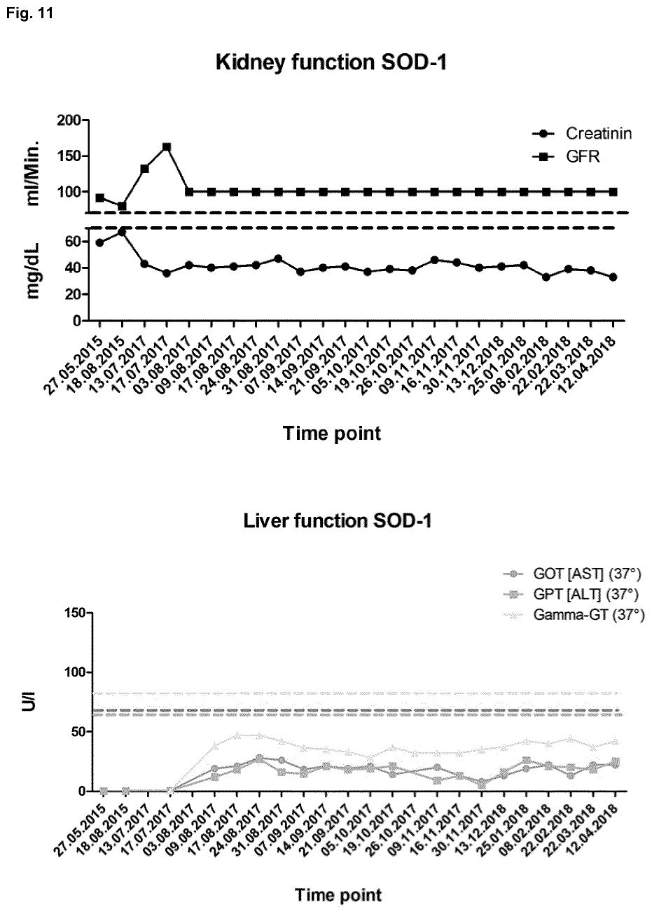

FIG. 10: Toxicity results from an TARDBP patient with ALS.

FIG. 11: Toxicity results from a SOD-1 patient with ALS.

FIG. 12: GA and DL reduce intracellular calcium.

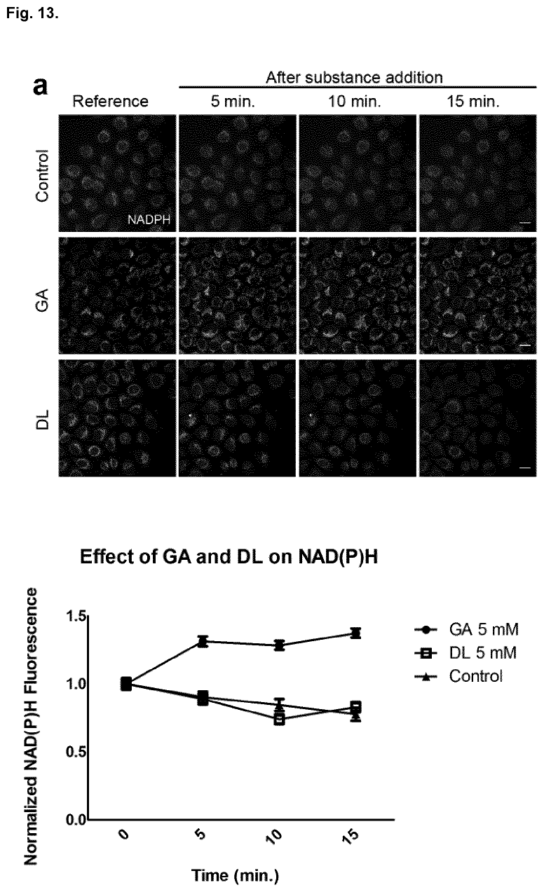

FIG. 13: GA increases mitochondrial NAD(P)H production.

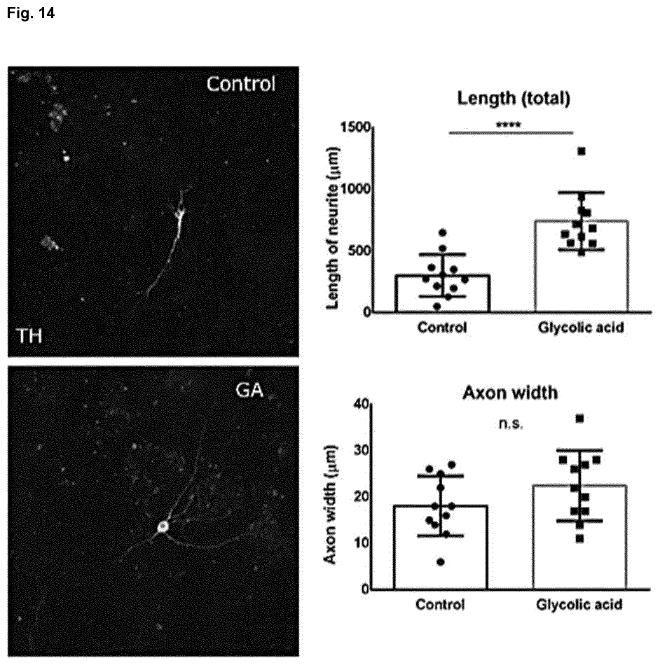

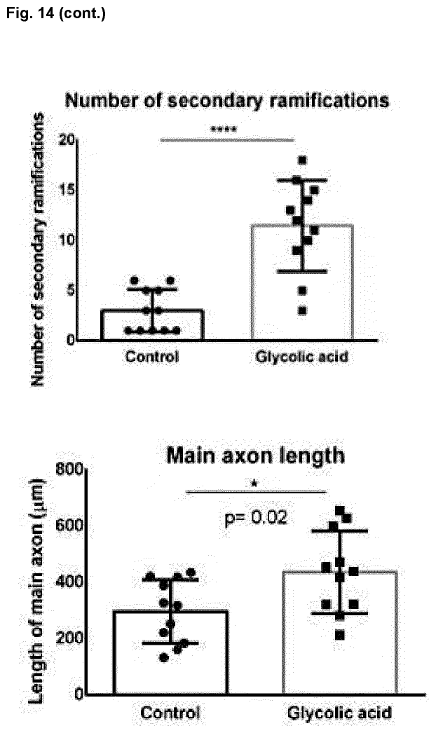

FIG. 14: Effect of GA treatment on the morphology of dopaminergic neurons.

FIG. 15: GA enhances SOCE and calcium influx during glutamate-triggered action potentials.

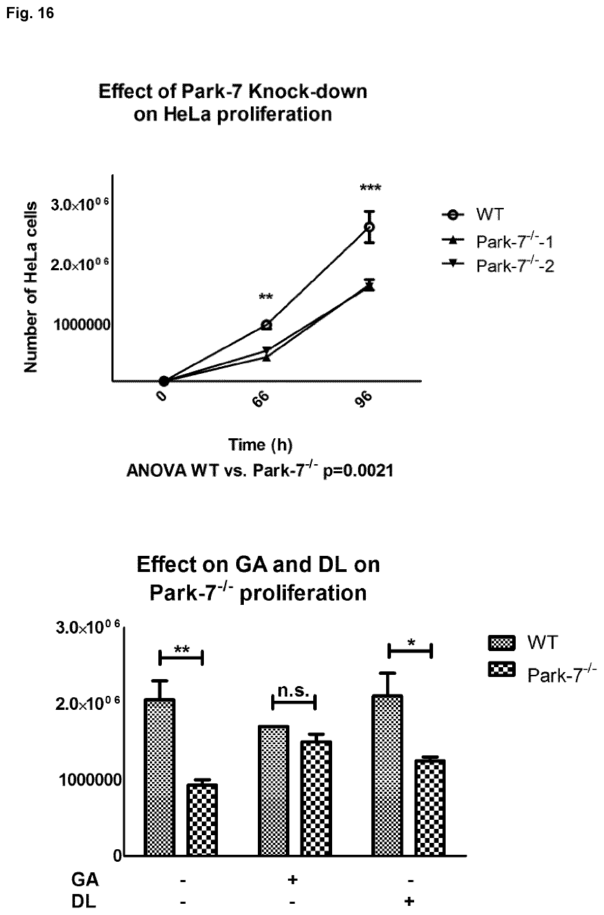

FIG. 16: GA but not DL rescues cell proliferation defects in PARK-7 −/− HeLa cells.

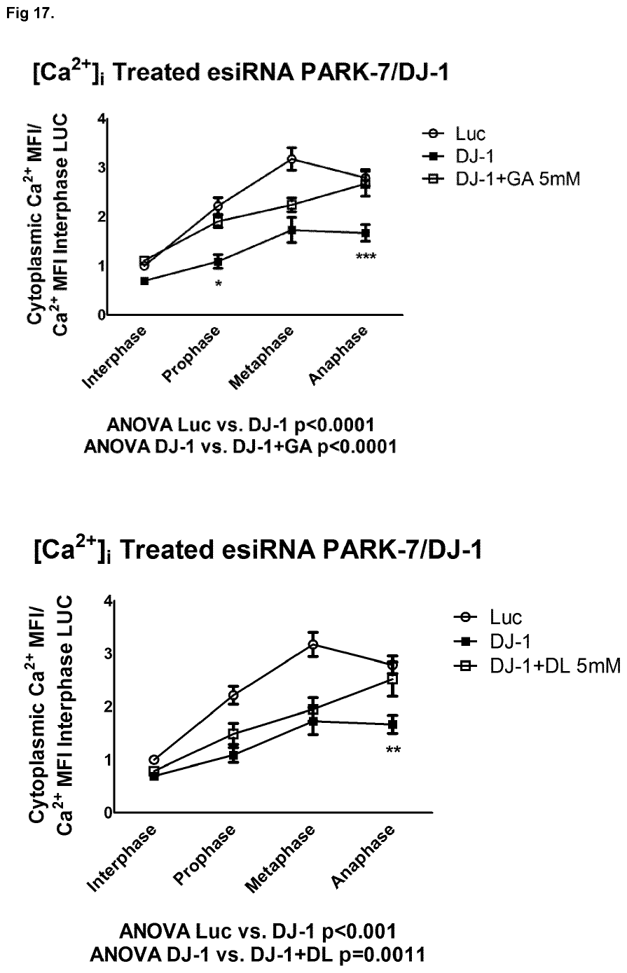

FIG. 17: GA enhances SOCE and calcium influx during mitosis in the absence of PARK-7/DJ-1.

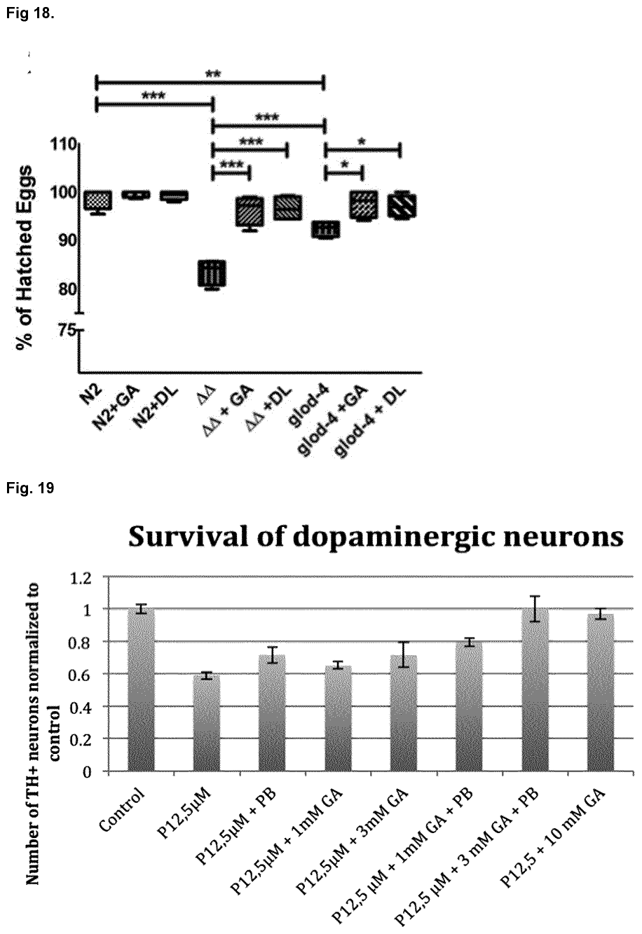

FIG. 18: GA and DL rescue embryonic lethality in djr1.1/djr1.2 and glod-4 KO C. elegans.

FIG. 19: GA combined with PB is more effective than GA alone in protecting the toxic effect of Paraquat on dopaminergic neurons.

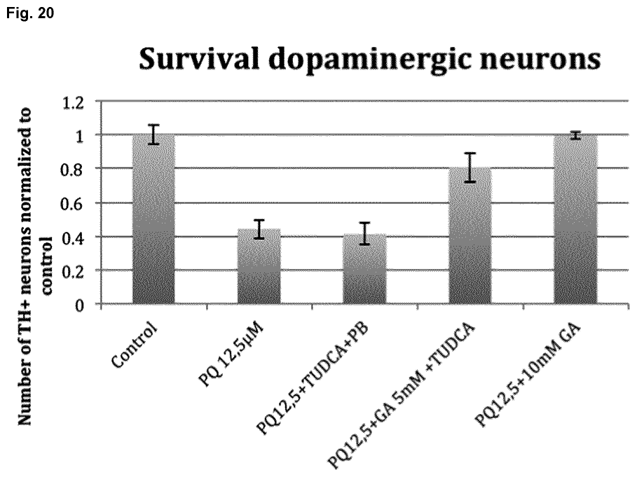

FIG. 20: GA combined with TUDCA is more effective than PB combined with TUDCA in protecting the toxic effect of Paraquat on dopaminergic neurons.

### DETAILED DESCRIPTION OF THE FIGURES

FIG. 1: GA combined with LA is more effective than GA alone in protecting the toxic effect of Paraquat on dopaminergic neurons.

Dopaminergic neurons were isolated and plated at a concentration of 1.000.000 cells/ml (100 μl/well) in a 96 well plate and cultured in medium with one or more of the various factors indicated, as described in the examples below. The survival of dopaminergic neurons in the presence and absence of various agents of the invention, either with or without paraquat challenge, is shown by the number of TH positive neurons normalized to the control treatment.

FIG. 2: Liver Function: Individualised clinical trial data from a FUS patient with ALS.

Results of blood analyses performed once a week to every two weeks showing the concentration of hepatic enzymes before (24 Mar. 2017) and after administration of Glycolic acid and D-lactate. The peak on the 24 May 2017 is due to an infection as can be observed by the increase of the C-reactive protein on the same day (FIG. 4).

FIG. 3: Kidney function: Individualised clinical trial data from a FUS patient with ALS

Results of blood analyses performed once a week to every two weeks showing the concentration of creatinin (a waste substance washed away by the liver used as a biomarker of kidney function) and the values of the glomerular flow rate (also a marker of renal function) before (24 Mar. 2017) and after administration of Glycolic acid and D-lactate.

FIG. 4: Creatine Kinase: Individualised clinical trial data from a FUS patient with ALS

Results of blood analyses performed once a week to every two weeks showing the concentration of creatine kinase (an enzyme released up muscle destruction) before (24 Mar. 2017) and after administration of Glycolic acid and D-lactate.

FIG. 5: Gripping Force: Individualised clinical trial data from a FUS patient with ALS

Gripping force measured in kilograms with the help of a Digital Hand Dynamometer once a week to every two weeks. The results show a 25% decrease until just before the target dose in reached with a posterior stabilization of the force.

FIG. 6: Muscle Strength Arm: Individualised clinical trial data from a FUS patient with ALS

Evolution of the muscle strength on the right arm was measured once a week to every two weeks using the Janda Muscle Strength Scale. Treatment with glycolic acid and D-lactate together stabilized the muscle strength thereby delaying the progression of the disease. This can be clearly observed for the upper arm, where a clear drop within the first three months of Treatment with glycolic acid and D-lactate together stabilized the muscle strength thereby delaying the progression of the disease. This can be clearly observed for the upper arm, where a clear drop within the first three months of 2017 occurred and was stabilized the next 6 months after the target dose with the medication was reached.

FIG. 7: Muscle Strength Leg: Individualised clinical trial data from a FUS patient with ALS

As a reference in the same patient evolution of the muscle strength on the right and left legs measured in the routine controls before the treatment started using the Janda Muscle Strength Scale. Upper graphic shows values for the left leg. Lower graphic shows values for the right leg. As it can be observed, in the absence of treatment, the muscle strength in the legs of the patient already dramatically dropped within the first three months and only a muscle contraction without any movement of the limb (⅕) could be observed in many muscles 7 months after the first examination.

FIG. 8: Pharmacokinetics: Blood concentration of GA after administration

The concentration of GA in the blood of a subject post-administration is shown in the figure. As can be observed, GA levels reach 120 mg/L in the blood 1-hour post-administration and reduce to approx. 40 or 20 mg/I after 2- or 3-hours post-administration, respectively. As can also be observed, DL levels reach 140 mg/L in the blood 1-hour post-administration and reduce to approx. 20 mg/I after 2- or 3-hours post-administration.

FIG. 9: CSF concentration of GA after administration

The concentration of GA in the CSF of a subject post-administration is shown in the figure. As can be observed, GA levels are approximately 20 mg/I in the CSF 1-hour post-administration. As can also be observed, DL levels are approximately 5 mg/I in the CSF 1-hour post-administration.

FIG. 10: Toxicity results from an TARDBP patient with ALS

In analogy to FIGS. 2 and 3, kidney and liver function was assessed during administration of GA and DL according to scheme presented in the examples. The Creatine and GFR levels indicate no toxicity to the kidney. The GOT, GPT and Gamma GT values indicate no toxicity to the liver.

FIG. 11: Toxicity results from a SOD-1 patient with ALS

In analogy to FIGS. 2 and 3, kidney and liver function was assessed during administration of GA and DL according to scheme presented in the examples. The Creatine and GFR levels indicate no toxicity to the kidney. The GOT, GPT and Gamma GT values indicate no toxicity to the liver.

FIG. 12: GA and DL reduce intracellular calcium

GA and DL reduce intracellular calcium. HeLa cells were loaded with Fluo4-AM and fluorescence was monitored with the help of a fluorescent plate reader. Values are normalized to the initial fluorescent value.

FIG. 13: GA increases mitochondrial NAD(P)H production 5 mM GA but not DL increases mitochondrial NAD(P)H production. NAD(P)H levels were measured with the help of a UV confocal microscope as described (ex. 350 nm, em. 460±25 nm, Blacker et al 2014). All values were referenced to the value obtained before substance addition.

FIG. 14: Effect of GA treatment on the morphology of dopaminergic neurons

Effect of GA treatment on the morphology of dopaminergic neurons. Fluorescent microscopy images on the left show to TH+ neurons in a primary mesencephalic cell culture with (GA) and without (Control) treatment. 5 mM GA increases the length of the neurites and the main axon and the number of secondary ramifications. FIG. 16:

FIG. 15: GA enhances SOCE and calcium influx during glutamate-triggered action potentials

GA enhances SOCE and calcium influx during glutamate-triggered action potentials. Fluorescent microscopy images in a show the effect of calcium, glutamate and ionomycin on intracellular calcium in Fluo-4 AM charged cortical neurons at different time points. Graphic in b shows the variations with time and after addition of calcium (SOCE), glutamate (action potential) and ionomycin in GA treated and control Fluo-4 AM charged cortical neurons. Box-plot graphic in c shows the total amount of calcium (area under the curve) entering the neuron after the addition of calcium to the media in control and 2.5 mM GA treated neurons. Box-plot graphic in d shows the total amount of calcium (area under the curve) entering the neuron after the addition of glutamate to trigger an action potential in control and 2.5 mM GA treated neurons.

FIG. 16: GA but not DL rescues cell proliferation defects in PARK-7 −/− HeLa cells

GA enhances cell proliferation in PARK-7 −/− HeLa cells. Left graphic shows the quantification of cell number up to 96 hours after plating HeLa cells. Knocking-down PARK-7 with CRISP/Cas-9 leads to a reduced cell proliferation when compared to WT cells. Right graphic shows the number of cells after 48 hours with and without GA or DL treatment. Treatment with GA increases cell proliferation in HeLa cells.

FIG. 17: GA enhances SOCE and calcium influx during mitosis in the absence of PARK-7/DJ-1

HeLa cells were loaded with Fluo-4 AM, a dye used to measure calcium concentration in living cells, as described by the manufacturer and recorded for 4 hours. Graphics show the variations in intracellular calcium concentration during mitosis in WT and cells treated with siRNA against PARK-7/DJ-1 to down-regulate this gene. Down-regulation of this gene leads to a decrease calcium influx during mitosis and GA (left graphic) and DL (right graphic) were able to rescue this phenotype.

FIG. 18: GA and DL rescue embryonic lethality in djr1.1/djr1.2 and glod-4 KO C. elegans

Graphic showing the percentage of hatched eggs in the different C. elegans strains. Knocking down djr1.1/djr1.2 or glod-4 leads to a reduction an increase in embryonic lethality shown as a decrease in the percentage of hatched eggs. Feeding the worms with GA or DL led to a rescue of embryonic lethality.

FIG. 19: GA combined with PB is more effective than GA alone in protecting the toxic effect of Paraquat on dopaminergic neurons.

Dopaminergic neurons were isolated and plated at a concentration of 1.000.000 cells/ml (100 μl/well) in a 96 well plate and cultured in medium with one or more of the various factors indicated, as described in the examples below. The survival of dopaminergic neurons in the presence and absence of various agents of the invention, either with or without paraquat challenge, is shown by the number of TH positive neurons normalized to the control treatment.

FIG. 20: GA combined with TUDCA is more effective than PB combined with TUDCA in protecting the toxic effect of Paraquat on dopaminergic neurons.

Dopaminergic neurons were isolated and plated at a concentration of 1.000.000 cells/ml (100 μl/well) in a 96 well plate and cultured in medium with one or more of the various factors indicated, as described in the examples below. The survival of dopaminergic neurons in the presence and absence of various agents of the invention, either with or without paraquat challenge, is shown by the number of TH positive neurons normalized to the control treatment.

### EXAMPLES

The invention is further described by the following examples. These are not intended to limit the scope of the invention.

### Example 1: Treatment of Dopaminergic Neurons

Dopaminergic neurons were isolated and plated at a concentration of 1.000.000 cells/ml (100 μl/well) in a 96 well plate. After 3-4 hours incubation at 37° C., 20 μl of medium was removed from each well. (VF=80 μl). Changes in medium and start point of the treatment were done in the following day in vitro (DIV). The following protocol was employed in order to assess the survival of dopaminergic neurons in the presence and absence of various agents of the invention combination, either with or without paraquat challenge.
DIV.1: Change half of medium N2 (40 μl) with fresh medium N2.
DIV.3: Change half of the medium and start with medium (control) or with medium A containing LA (different concentrations) and/or GA (normally 5 mM or 10 mM). Medium A is N2 medium but without FBS and N2-Supplement.
DIV.5: Second round of control or treatment with LA and/or GA. Half of the medium (40 μl) was replaced by fresh medium A with the different agents.
DIV.7: Paraquat 25 μM treatment starts with or without GA and L-alanine. Half of the medium (40 μl) was replaced by fresh medium A with the different treatment combinations or without (control).
DIV.9: Second day of treatment with Paraquat (PQ) 25 μM in addition to the other substances (LA and GA).
DIV.10: Fixation 2% of PFA during 20 min at 37° C. or overnight at 4° C.

Results:

As can be seen from FIG. 1, PQ treatment leads to a severe reduction in neuron survival. The addition of 0.01 mM LA alone with PQ provides no rescue. The addition of 5 mM GA in combination with PQ treatment leads to a rescue over PQ treatment alone. Surprisingly, the addition of 0.01 mM LA to 5 mM GA in PQ treatment provides an unexpected enhancement of GA rescue of the PQ induced neuronal death. The use of 0.1 mM LA shows an even greater enhancement of GA-induced recovery, although at 10 mM GA the PQ-induced challenge is rescued completely, such that no LA induced enhancement is observed.

### Example 2: Clinical Treatment in a Patient with ALS

The following treatment scheme was established for patients in individualised clinical trials (according to § 4 AMG—German medical drug legislation). Patients with ALS were recruited for the study and agreed to all legal regulations surrounding the curative attempt. Potential side effects were closely monitored. Liver and kidney values were checked once a week (1 day after dosing), in order to monitor whether any unwanted side effects were observed. To test the therapeutic effect, the evolution of the ALS score and strength evolution were measured over time.

Treatment Scheme:
1st week: D-lactic acid 20 mg/kg body weight (BVV)
2nd week: D-lactic acid 40 mg/kg BW+glycolic acid 20 mg/kg BW
3rd week: D-lactic acid 40 mg/kg BW+glycolic acid 40 mg/kg BW
4th week: D-lactic acid 60 mg/kg BW+glycolic acid 40 mg/kg BW
5th week: D-lactic acid 60 mg/kg BW+glycolic acid 60 mg/kg BW
6th week: D-lactic acid 80 mg/kg BW+glycolic acid 60 mg/kg BW
7th week: D-lactic acid 80 mg/kg BW+glycolic acid 80 mg/kg BW
8th week: D-lactic acid 100 mg/kg BW+glycolic acid 80 mg/kg BW
9th week: D-lactic acid 100 mg/kg BW+glycolic acid 100 mg/kg BW
10th week: D-lactic acid 120 mg/kg BW+glycolic acid 100 mg/kg BW
11th week: D-lactic acid 120 mg/kg BW+glycolic acid 120 mg/kg BW
12th week: D-lactic acid 140 mg/kg BW+glycolic acid 120 mg/kg BW
13th week: D-lactic acid 140 mg/kg BW+glycolic acid 120 mg/kg BW
14th week: D-lactic acid 140 mg/kg BW+glycolic acid 140 mg/kg BW
15th week: D-lactic acid 160 mg/kg BW+glycolic acid 160 mg/kg BW

All patients also received 6 grams of L-Alanine per day.

The above-mentioned treatment regime was conducted in 4 patients, either with FUS, TARDBP or SOD-1 mutations underlying their ALS. After week 15, the treatment was continued at D-lactic acid between 100-120 mg/kg BW+glycolic acid 100-120 mg/kg BW depending on the patient due to the undesired intestinal side-effects. The patients were treated between 4 months and 17 months.

The patients received the GA and DL in a 20% solution diluted in apple juice, with pH adjusted to approximately 7, and the LA as a tablet.

Results:

As can be seen from FIGS. 2 and 3, no significant change in kidney or liver function is evident due to the treatment over a time period of up to 17 months.

From these measurements, we conclude that the administration of 100-120 mg/kg of glycolic acid and D-lactate together is not toxic, does not affect the immune system and does not cause an autoimmune reaction.

Further experiments were undertaken with the help of a Digital Hand Dynamometer to determine creatine kinase levels in blood from the patients. Creatine kinase is an enzyme released upon muscle destruction. AS can be observed from FIG. 4, creatine kinase is released in ever decreasing amounts during the course of the treatment, thereby indicating that muscle destructions is being slowed or prevented. The administration of 100-120 mg/kg of glycolic acid and D-lactate together therefore reduces muscle destruction.

Further experiments were undertaken to determine gripping force in patients during the course of the treatment. As is shown in FIG. 5, the treatment leads to a clear slowing of the reduction in gripping force in both left and right hands. The red line presented in FIG. 5 indicates the usual rate of gripping force reduction observed in patients without receiving the treatment, as described herein.

Further experiments were undertaken to determine muscle strength on the right arm measured using the Janda Muscle Strength Scale. As is shown in FIG. 6, the treatment leads to a clear slowing of the reduction in muscle strength in the right upper arm. The progression of the disease thereby appears to be delayed by the administration of the combination employed.

In the same patient, evolution of the muscle strength on the right and left legs was measured in routine controls before the treatment started, using the Janda Muscle Strength Scale. As can be observed in FIG. 7, in the absence of treatment, the muscle strength in the legs of the patient already dramatically dropped within the first three months and only a muscle contraction without any movement of the limb (⅕) could be observed in many muscles 7 months after the first examination. This again speaks for the therapeutic efficacy of the treatment, when comparing the delay in disease progression shown in FIGS. 4-6 and the disease progression in FIG. 7.

Preliminary pharmacokinetic analyses were undertaken in order to determine whether the GA and DL administered to the patients orally were absorbed into the blood stream and into the CSF. As can be seen from FIG. 8, GA levels reached 120 mg/L in the blood 1-hour post-administration and were reduced to approx. 40 or 20 mg/I after 2- or 3-hours post-administration, respectively. As can also be observed, DL levels reach 140 mg/L in the blood 1-hour post-administration and are reduced to approx. 20 mg/I after 2- or 3-hours post-administration.

As can be observed in FIG. 9, GA levels are approximately 20 mg/I in the CSF 1-hour post-administration. As can also be observed, DL levels are approximately 5 mg/I in the CSF 1-hour post-administration. 100 mg/kg GA and 100 mg/kg DL was administered in patients to obtain the pharmacokinetic data.

Additional experimental results are provided for the additional ALS patients with SOD-1 and TARDBP mutations as the underlying genetic background to their ALS (FIGS. 10 and 11). Similar to FIGS. 2 and 3, kidney and liver function was assessed during administration of GA and DL according to scheme presented herein. The Creatine and GFR levels indicate no toxicity to the kidney. The GOT, GPT and Gamma GT values indicate no toxicity to the liver.

These results indicate that the combination of GA with AL leads to a therapeutic improvement in a clinical setting, by slowing disease progression in ALS patients, using various functional and molecular readouts. Furthermore, the use of AL appears to avoid any unwanted side effects or reductions in function of the kidney or liver in patients receiving the inventive treatment over approximately 15 months. The present invention is therefore defined by a combination of key advances and advantages in the treatment of neurological disease, whereby the combination of GA with AL shows not only functional improvement but also voids the side effects suggested to occur in long term GA administration, such as kidney disfunction, or DL administration in high doses such D-lactate acidosis that induces neurological symptoms such as delirium, ataxia, and slurred speech.

### Example 3: Effect of Glycolic Acid and D-Lactate on Neurons and Neuronal Plasticity

In earlier studies, the inventor found that glycolic acid (GA) and D-lactate (DL) protect mitochondrial function thereby protecting dopaminergic neurons against environmental toxins in an in vitro model of Parkinson's disease. We have now investigated the effects of both substances at the cellular level and tested their therapeutic potential in other neurological conditions, like ALS or stroke. Our preliminary results show that GA but not DL reduce intracellular calcium and enhance energy production (NAD(P)H) in HeLa cells and neurons (see FIGS. 12 and 13).

We also observed a positive trophic effect on neuronal morphology. In dopaminergic neurons, glycolic acid led to increases in neurite formation with increased length of neurites and axons and increased secondary ramifications (FIG. 14). Using calcium imaging on cortical neurons, we also analysed the effects of GA on calcium transients and calcium influx during the action potential. Our results show that cortical neurons treated with GA have bigger calcium transients, increased storage operated calcium entry (SOCE) and higher increases in intracellular calcium during the action potential (FIG. 15). Altogether, these results suggest that glycolic acid and to a lesser extent, D-lactate, could partially revert the effects of aging and enhance neuroplasticity.

Several other studies have investigated the effect of psychotherapy-like approaches in psychiatric animal models. Extinction of conditioned fear has been successfully used in a post-traumatic stress disorder (PTSD). Extinction of conditioned fear bears resemblance to one form of cognitive therapy, exposure therapy. It has also been shown that variations in the expression of Tcf4 lead to a cognition/plasticity phenotype similar to the one observed in schizophrenic patients. Interestingly, these mice also show a higher susceptibility to negative external cues like social defeat and isolation rearing. Putting these mice in an enriched environment (in the case of isolated mice) and increasing handling care (in the case of social defeat) can ameliorate the symptoms caused by both negative cues.

By employing these models, we can assess GA and the combinations of the invention in their ability to increase neuronal plasticity, and potentially their effect in enhancing a recovery from schizophrenia like phenotypes in animal models, thereby potentially improving the positive effects of psychotherapy, for application in other mammal, such as human subjects.

Cortical and Dopaminergic Primary Neuronal Cell Cultures

Primary cortical neuronal cell cultures were prepared from E15.5 embryos. Briefly, brain cortex from E15.5 pregnant wild type C57Bl/6J or PARK-7^{-/-} mice were dissected and placed in cold HBSS without Ca2+ and Mg2+(Sigma Aldrich H6648, Germany, EU). Once freed from all other cerebral structures, cortex were placed in an empty petri dish, sliced with the help of a scalpel and trypsinized using a 1:1 mixture of Trypsin (Gibco 25200-056):HBSS at 37° C. for 7 min. The samples were then centrifuged for 4 min. at 800 rpm and the supernatant was replaced with plating medium (89% Neurobasal A, 8.9% FBS, 0.9% L-glutamine, 0.9% N2 supplement and 0.4% P/S). After mechanical dissociation with the help of a fire-polished Pasteur pipette, the number of cells per ml was estimated under the microscope with the help of a Neubauer Chamber, and cortical neurons were plated at a density of 65,000 cells per well in 96-well Greiner plates (Greiner Bio-one 655090, Germany, EU), coated with Poly-L-Lysine (100 μg/ml, Sigma Aldrich P6282, Germany, EU) and maintained at 37° C. and 5% CO2. 4 hours after plating, all the medium was changed to culture medium (96.7% Neurobasal A, 0.9% L-glutamine, 1.9% B-27, 0.4% P/S). 50% of the culture medium was changed every 3 days.

Primary mesencephalic neuronal cell cultures were prepared as previously described. Briefly, E14.5 embryos were obtained from C57JBL6 pregnant mice after cervical dislocation. Brain mesencephali were dissected under the microscope and digested with Trypsin-EDTA 0.12% (Life Technologies, USA) for 7 min. The trypsin reaction was then stopped by adding basic medium (BM), containing Neurobasal A medium (Gibco, USA), 1 mg/mL Pen/Strep, 10% FCS, and 200 mM L-Glutamine, and cells were mechanically dissociated using a fire-polished Pasteur pipette. Medium was fully replaced after 5 min, centrifugation at 1200 rpm, aspiring the supernatant and adding 8 mL of fresh BM to the pellet. Concentration of cells in the medium was estimated using a Neubauer chamber and a 100 μL of medium containing 106 cells/mL plated per well in a 96-well plate (Greiner Sensoplate, Germany, EU). Then a 20 μL of medium was removed from the well and 24 h later, ⅓ of the media was replaced with fresh BM. On differentiation day 3 (DIV3) and DIV5, half of the medium was replaced with B27 medium, containing Neurobasal A medium, 1 mg/mL Pen/Strep, 200 mM L-Glutamine, and B-27 supplement.

Assessment of the Effect of GA and DL on Dopaminergic Neurons Morphology

Treatment with vehicle (distilled water), 10 mM GA or 10 mM DL were administered on DIV3 and DIV 9 and cells were fixed on DIV10. The effect of GA and DL on dopaminergic neurons was assessed through semi-automatic quantification of neurite length and width of TH+ neurons after treatment. Briefly, neurons were fixed using 4% paraformaldehyde for immunocytochemical analysis after treatment. Dopaminergic TH⁺ neurons were observed using an inverted fluorescence microscope (Olympus) under a 20× objective.

Calcium Imaging on Cortical Neurons

On DIV7 cultures were rinsed once with HBSS without Ca²⁺ and Mg²⁺, and incubated in 2 μM Fluor 4-AM (Life Technologies F14201, Paisley, UK) in HBSS at a 1:1000 dilution, previously dissolved in anhydrous DMSO (Sigma Aldrich 276855, Germany, EU) and Pluronic F-127 (Sigma Aldrich P2443, Germany, EU), for 45 min. at 37° C. and 5% CO₂. After the incubation, samples were washed for 5 min. with HBSS, and then incubated in a mixture of HBSS and HEPES 5 mM (Sigma Aldrich H0887, Germany, EU), with or without GA, DL for 25 min. before starting the experiments. An inverted Olympus IX50 microscope with ex/em filters of 488/510 nm was used to record live imaging at a constant temperature using the FView Soft Imaging System. Neurons were then sequentially treated with 1.8 mM CaCl₂), 300 μM of Glutamic acid (Sigma Aldrich G8415, Germany, EU), and 2 μM lonomycin (Sigma Aldrich 10634, Germany, EU).

Image Analysis of Calcium Imaging on Primary Cortical Neurons

Variations in the Fluo-4 AM fluorescence during Ca²⁺ and/or glutamate addition were analyzed using FIJI Image Analysis Freeware. The ROIs were determined using the standard deviation function for the stack of images before and after the addition of 1.8 mM CaCl₂ (for changes in intracellular Ca²⁺) or before and after the addition of glutamate. When used on a time-lapse stack of images, this function allows the identification of those cells that react to the added substance by generating an image, where only cells that experienced a signal intensity difference are shown. Once all ROIs were identified and selected, the MFI of each ROI for each time-point was measured with the measure function of the program to generate a matrix with the raw MFI values for each ROI for each time point. This matrix was exported as an excel table and after background subtraction two types of normalization were done depending on the experiments. To determine the effect of GA and DL on Ca²⁺ influx after CaCl₂ addition, all ROI values were normalized to the initial value within that ROI (i.e. at time-point 0). To determine the effect of GA and DL on Ca²⁺ influx after CaCl₂ addition and after glutamate addition, all ROIs where normalized using a max-min normalization as previously described: ([Ca²⁺]_{Ca}—[Ca²⁺]ₜ₀/([Ca²⁺]_{ionomycine}-[Ca²⁺]ₜ₀) Once the new matrix with the normalized values was generated, we determined the area under the curve (AUC) in excel using the formula: (Y1+Y2)/2*(X2−X1). The AUC was then obtained as the sum of all the generated values.

NAD(P)H Live-Cell Microscopy on HeLa Cells

NAD(P)H live-cell microscopy on HeLa cells was performed as previously described. Briefly, NAD(P)H fluorescence intensity time series were performed on a ZEISS LSM880 inverted confocal equipped with an incubation chamber to maintain 37 Celsius degree and 5% of CO2. Fluorophores were excited by using a 355 nm UV laser (Coherent), while the fluorescent signal was detected using a GaAsP spectral detector narrowing down the band of absorption between 455 and 473 nm. In order to maximize the transmission efficiency of the system in excitation and detection and reduce the aberrations due to the watery environment, a ZEISS Plan C-ApoChromat 40×/1.2 Water lens with depth compensating correction collar was used. In addition, bright field images were taken by using a HeNe 633 laser as source of light and a T-PMT to detect the signal. The sampling factor in XY (pixel size) of each image was equal to 208 nm, which lead to a final resolution of approximately 600 nm. For each image a volume of 5 μm around the specimen central plane was taken by acquiring 3 planes separated by a Z-step of 2.5 μm.

Time series measurements were obtained with 5 min time resolution. Fluorescence intensity levels were extracted using FIJI Image Analysis Freeware

### Example 4: Effect of Glycolic Acid and D-Lactate on Mitosis and Embryonic Development

It has been shown that storage operated calcium entry and calcium influx is important for mitosis. We therefore tested whether DJ-1/PARK-7 lead to alterations in cell proliferation in HeLa cells and worms.

Determination of the Effect of GA and DL on Cell Growth

Cell growth was determined by two different methods. The first method (WST1-Assay) was used to analyze cell growth at different time points using the same plates: 500 cells of 8 different PARK7 KO clones and HeLa Kyoto wild type cells were seeded in 96 well plates (6 wells/line). For each time point (0 h, 48 h, 122 h, and 144 h), WST1 was added to the cells according to the manufacturers instructions and incubated for 30 min at 37° C. Absorbance was measured at 450 nm and 620 nm using an EnVision Plate Reader (PerkinElmer).

The second method was used to analyze the rescue effect of GA and DL. Briefly, HeLa cells were seeded and treated with medium containing distilled water, 5 mM GA, or 5 mM DL. 48 hours later, the number of living cells was calculated with the help of an automated cell counter (ThermoFischer, USA).

CRISPR

HeLa-Kyoto PARK7 KO clones had been kindly provided by Martin Stewart (Koch Institute, MIT, Cambridge, USA). Briefly, cells were electroporated with the NEON device (Invitrogen) using a sgRNA-Cas9-NLS complex targeting human PARK7 at exon 1. Subsequently, cells were seeded in clonal dilution and clones were characterized by genotyping, sequencing, and Western blot.

Determination of Embrionic Lethality in C. elegans

All C. elegans strains were maintained on NGM agar plates seeded with Escherichia coli NA22 at 15° C. Wild type (N2) and mutant strains AAdjr and glod-4(tm1266) were obtained from Prof. Kurzchalia's laboratory at the Max Planck Institute for Cell Biology and Genetics. The procedures to obtain the DJ-1 double mutant mice has been already described [3]. To determine embryonic lethality, individual adult worms from each strain (with or without GA or DL treatment) were transferred to a 6 well-plate well with NGM and E. coli (NA22) (with or without GA or DL) to lay eggs. After 4 hours, adult worms were removed and the number of laid eggs was counted. The percentage of hatched eggs was calculated (L1/(L1+remaining eggs)*100) 8 hours after removing the adults.

Determination of the Effect of GA and DL on Calcium Influx During Mitosis in HeLa Cells

HeLa-Kyoto cells stably expressing histone H2B-mCherry and mouse DJ-1 were used. Cells were maintained in DMEM supplemented with 10% fetal bovine serum (FBS), 2 mM GlutaMAX, 100 unit/ml penicillin, 100 μg/ml streptomycin. For esiRNA treatment, cells were plated at a density of 15.000 cells/well in an ibidi 8 well chamber (Cat. no 80826, ibidi, Germany, EU), transfected with different esiRNAs (RLUC as empty vector, hPARK-7 and hKIF11 as positive control) (all esiRNAs were obtained from Eupheria, Germany, EU), and left for 72 hours before performing calcium imaging. esiRNA transfection was performed as follows. esiRNA was diluted in distilled water to a concentration of 20 ng/μl. For each well, two solutions were made: 1. 50 μl containing OptiMEM (49.2 μl) and RNAiMax (0.8 μl) and 2. 50 μl containing OptiMEM (46.5 μl) and 70 ng of esiRNA (3.5 μl). Both solutions were mixed 1:1, added to the well and incubated for 20 min. at RT. 150 μl of medium without antibiotics containing 15,000 HeLa cells were added on top and gently mixed. Cells were then place in the incubator for a minimum of 8 hours. After this time, media was changed for normal media.

Calcium Imaging on HeLa Cells During Mitosis

WT or esiRNA-treated HeLa-Kyoto cells plated on 8 well ibidi p-Slide cell culture chambers (Ibidi, Germany, EU) were gently washed with PBS (no Ca2+, 2 mM glucose), incubated with 2 μM Fluo-4 AM (1:1000 dilution) in PBS (no Ca2+, 2 mM glucose) for 30 min., washed 5 min. with PBS without Ca2+ and washed with PBS containing Ca2+(with or without 5 mM of GA or DL or the different calcium blockers) for 20 min. Cells were then imaged using a Deltavision fluorescent microscope (GE Healthcare, USA) with ex/em filters of 475/523 nm for Fluo-4 AM and 575/632 nm for H2B-mCherry for 4 hours under constant temperature (37° C.) and atmospheric CO2 (5%). In total, 10 positions per well were selected and pictures of each field in both wavelengths were obtained every 15 min.

Image Analysis of Calcium Fluorescence During Mitosis

We observed that the Fluo-4 AM dye started to leak out of the cells into the medium after 1.5 hours of imaging. Therefore, to measure changes in the intracellular [Ca2+] in HeLa cells, we only used images from the first hour of the time-lapse video. Images were analyzed using FIJI Image Analysis Freeware (https://fiji.sc). MFI of the Fluo-4 AM signal within the cell was determined using manually selected ROIs covering the whole cell area for each time-point. After background subtraction, each MFI value was assigned to a certain mitotic phase using the H2B-mCherry signal to determine the mitotic phase of that cell. All values obtained were then normalized to the mean MFI obtained from cells in interphase in the control group (either WT or cells treated with RLUC esiRNA).

Mitosis duration was analyzed by counting the number of video frames needed (4 frames per hour) to go from prophase to anaphase and multiplying this number by 15 minutes.

### Example 5: Treatment of Dopaminergic Neurons with a Combination of Glycolic Acid and PB, or Glycolic Acid and TUDCA

Dopaminergic neurons were isolated and plated at a concentration of 1.000.000 cells/ml (100 μl/well) in a 96 well plate. After 3-4 hours incubation at 37° C., 20 μl of medium was removed from each well. (VF=80 μl). Changes in medium and start point of the treatment were on the following day in vitro (DIV). The following protocol was employed to assess the survival of dopaminergic neurons in the presence and absence of various agents of the inventive combination, either with or without paraquat challenge.
DIV.1: Change half of medium N2 (40 μl) with fresh medium N2.
DIV.3: Change half of the medium and start with medium (control) or with medium A containing PB (0.15 mM) and TUDCA (0.5 mM), or with Medium A containing GA (normally 1 mM, 3 mM, or 10 mM) or with Medium A containing GA (1 mM or 3 mM) and PB (0.15 mM) or with Medium A containing GA (5 mM) and TUDCA (0.5 mM) or with Medium A containing PB (0.15 mM). Medium A is N2 medium but without FBS and N2-Supplement.
DIV.5: Second round of control or treatment with different treatments. Half of the medium (40 μl) was replaced by fresh medium A with the different agents.
DIV.7: Paraquat 12.5 μM treatment starts alone or in combination with the treatments explained above. Half of the medium (40 μl) was replaced by fresh medium A with the different treatment combinations or without (control).
DIV.9: Second day of treatment with Paraquat (PQ) 12.5 μM in addition to the other treatments as explained above.
DIV.11: Fixation 2% of PFA during 20 min at 37° C. or overnight at 4° C.

The effect of the different treatments on dopaminergic neurons survival upon exposure to paraquat was assessed through of TH+ neurons after treatment. Briefly, neurons were fixed using 2% paraformaldehyde for immunocytochemical analysis after treatment. Dopaminergic TH⁺ neurons per well were identified and counted using an inverted fluorescence microscope (Olympus) under a 20× objective.

Results:

As can be seen from FIG. 19, treatment with 12.5 μM of PQ leads to a reduction in neuron survival. The addition of 0.15 mM PB alone with PQ provides a certain rescue (PQ:0.58 vs PQ+PB: 0.72, p=0.04). The addition of 1 mM GA alone with PQ provides no significant rescue (PQ:0.58 vs. PQ+1mMGA:0.65, p=0.08) and the addition of 3 mM GA in combination with PQ treatment leads to a non-significant rescue over PQ treatment alone (PQ:0.58 vs. PQ+3mMGA:0.71, p=0.13).

Surprisingly, the addition of 0.15 mM PB to 1 mM and 3 mM GA in PQ treatment provides an unexpected enhancement of GA rescue of the PQ induced neuronal death (PQ+1mMGA:0.65 vs. PQ+1mMGA+0.15 mM PB:0.79, p=0.02; PQ+3mMGA:0.71 vs. PQ+3mMGA+0.15mMPB:1, p=0.027). The use of 0.15 mM PB shows an enhancement of GA-induced recovery, thus reducing the concentrations of GA used to exert the same effect as 10 mM GA, to only 3 mM GA.

As can be seen from FIG. 20, treatment with 12.5 μM PQ leads to a reduction in neuron survival. The addition of 0.15 mM PB in combination with 0.5 mM TUDCA provides no rescue (PQ:0.44 vs. PQ+0.15 mM PB+0.5 mM TUDCA:0.41, p=0.74). Whereas PB does not increase the effect of TUDCA, 5 mM GA enhances the effect of TUDCA (PQ+0.15 mM PB+PQ+0.5 mM TUDCA:0.41 vs. PQ+5 mM GA+0.5 mM TUDCA:0.8, p=0.01).

1. A pharmaceutical combination, comprising:
glycolic acid or a pharmaceutically acceptable salt or ester thereof; and
L-Alanine or a pharmaceutically acceptable salt thereof.

2. The pharmaceutical combination according to claim 1, further comprising D-lactate or a pharmaceutically acceptable salt thereof.

3. The pharmaceutical combination according to claim 1, wherein:
glycolic acid or a pharmaceutically acceptable salt or ester thereof is in a pharmaceutical composition in admixture with a pharmaceutically acceptable carrier, and L-alanine or a pharmaceutically acceptable salt thereof, is in a separate pharmaceutical composition in admixture with a pharmaceutically acceptable carrier, or
glycolic acid and L-alanine, or pharmaceutically acceptable salts or esters thereof, are present in a kit, in spatial proximity but in separate containers and/or compositions, or
glycolic acid and L-alanine, or pharmaceutically acceptable salts or esters thereof, are combined in a single pharmaceutical composition in admixture with a pharmaceutically acceptable carrier.

4. (canceled)

5. (canceled)

6. The pharmaceutical combination according to claim 1, further comprising phenylbutyrate or a pharmaceutically acceptable salt or ester thereof and/or tauroursodeoxycholic acid or a pharmaceutically acceptable salt or ester thereof.

7. The pharmaceutical combination according to claim 1, wherein a composition comprises glycolic acid, and L-alanine, and is suitable for oral administration.

8. The pharmaceutical combination according to claim 1, wherein a composition comprises glycolic acid and L-alanine, and is suitable for injection.

9. The pharmaceutical combination according to claim 1, further comprising pyridoxine (Vitamine B6) and/or citrate.

10. The pharmaceutical combination according to claim 1, comprising a glycolic acid solution with 5-30 wt % glycolic acid.

11. The pharmaceutical combination according to claim 10, wherein the glycolic acid solution has a pH of 3 to 9.

12. The pharmaceutical combination according to claim 1, wherein glycolic acid and L-alanine have relative amounts of 100:1 to 1:10.

13. A method for the treatment or reducing the risk of a neurological medical condition comprising administering the pharmaceutical combination according to claim 1 to a subject in need thereof.

14. The method according to claim 13, wherein the neurodegenerative disease is Amyotrophic Lateral Sclerosis (ALS) or Parkinson's Disease.

15. A method for the treatment or reducing the risk of ischemic disease, comprising administering the pharmaceutical combination according to claim 1 to a subject in need thereof.

16. A method for the treatment or reducing the risk of male infertility and/or for enhancing sperm motility, comprising administering the pharmaceutical combination according to claim 1 to a subject in need thereof.

17. A method to stimulate neuronal plasticity, comprising administering the pharmaceutical combination according to claim 1 to a subject in need thereof.

18. A method to stimulate mitochondrial function and ATP production, comprising administering the pharmaceutical combination according to claim 1 to a subject in need thereof.

19. A method for the treatment or reducing the risk of an age-related medical condition associated with a decline in mitochondrial function, wherein said treatment and/or reducing risk comprises slowing, reversing and/or inhibiting the ageing process, the method comprising administering the pharmaceutical combination according to claim 1 to a subject in need thereof.

20. A method to stimulate the immune system, comprising administering the pharmaceutical combination according to claim 1 to a subject in need thereof.

21. A method to stimulate oocyte and fertility fitness, comprising administering the pharmaceutical combination according to claim 1 to a subject in need thereof.

22. The method according to claim 21 for the treatment or reducing the risk of disease- or age-related reduction in fertility in woman.

23. (canceled)

24. (canceled)
